(19) United States
(12) Patent Application Publication
(10) Pub. No.: US 2025/0325206 A1
(43) Pub. Date: Oct. 23, 2025

(54) SYSTEMS AND METHODS FOR PROVIDING THERAPY MANAGEMENT GUIDANCE FOR DIAGNOSIS AND MANAGEMENT OF KIDNEY DISEASE

(71) Applicant: Dexcom, Inc., San Diego, CA (US)

(72) Inventors: Matthew L. JOHNSON, San Diego, CA (US); Qi AN, San Diego, CA (US); John PADERI, San Francisco, CA (US); Rush BARTLETT, Austin, TX (US); Joshua Ray WINDMILLER, San Diego, CA (US); Christina CHEN, San Diego, CA (US)

(21) Appl. No.: 19/186,553

(22) Filed: Apr. 22, 2025

Related U.S. Application Data
(60) Provisional application No. 63/637,096, filed on Apr. 22, 2024.

(51) Int. Cl. A61B 5/1473; A61B 5/00; A61B 5/145; A61B 5/1495; A61B 5/20

(52) U.S. Cl. CPC A61B 5/1473; A61B 5/0004; A61B 5/0022; A61B 5/14532; A61B 5/1495; A61B 5/201; A61B 5/4833; A61B 5/4842; A61B 5/7225; A61B 5/7246

(57) ABSTRACT

Certain aspects of the present disclosure provide a monitoring system comprising a continuous analyte sensor configured to penetrate a skin of a patient and generate a sensor current indicative of analyte levels of the patient, and a sensor electronics module coupled to the continuous analyte sensor. The sensor electronics module comprises an analog to digital converter configured to receive the sensor current and convert the sensor current generated by the continuous analyte sensor into digital signals, a processor configured to convert the digital signals to a set of analyte measurements indicative of the analyte levels of the patient, and a Bluetooth antenna configured to transmit the set of analyte measurements wirelessly to a wireless communications device using Bluetooth or BLE communications protocols.

### CROSS-REFERENCE TO RELATED APPLICATIONS

This application claims priority to and benefit of U.S. Provisional Application No. 63/637,096, filed Apr. 22, 2024, which is hereby assigned to the assignee hereof and hereby expressly incorporated by reference in its entirety as if fully set forth below and for all applicable purposes.

### INTRODUCTION

The kidney is responsible for many critical functions within the human body, including filtering waste and excess fluids, which are excreted in urine, and removing acid that is produced by the cells of the body to maintain a healthy balance of water, salts, and minerals (e.g., such as sodium, calcium, phosphorus, and potassium) in the blood. In other words, the kidney plays a major role in homeostasis by renal mechanisms that transport and regulate water, salt, and mineral secretion, reabsorption, and excretion.

Kidney disease is generally classified as either acute or chronic based on the duration of the disease and/or whether the disease is caused by a specific event (e.g., dehydration, a medical procedure with toxic contrast or therapeutic, or significant surgery) or develops over time in response to a long-term disease. Chronic kidney disease (CKD) typically develops over time in response to a long-term disease such as high blood pressure or diabetes, for example, which slowly damages the kidneys and reduce their function over time, or more quickly in response to kidney damage that occurs acutely (e.g., as result of sepsis or AKI) but does not reverse. Symptoms of CKD develop slowly and may not be apparent until very little kidney function remains. A cute kidney injury (AKI) develops as a sudden decline in kidney function. The injury can be reversible in a short period of time but currently needs to be monitored in a hospital.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example therapy management system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3A illustrates example inputs and example metrics that are calculated based on the inputs for use by the therapy management system of FIG. 1, according to certain embodiments of the present disclosure.

FIG. 3B is a flow diagram depicting a method for training machine learning models to predict a patient's kidney disease state and/or kidney health, and provide recommendations to a patient based on the disease state, according to certain embodiments of the present disclosure.

FIG. 4A describes an example method for providing kidney disease therapy management guidance using an analyte monitoring system configured to measure at least 1,5-Anhydroglucitol (1,5-AG) levels, according to certain embodiments of the present disclosure.

FIG. 4B is a flow diagram illustrating an example method 401 for providing guidance to a patient in order to calculate the patient's reabsorption threshold, according to certain embodiments of the present disclosure.

FIG. 5 describes an example method for determining a filtration score and providing kidney disease therapy management guidance using an analyte monitoring system configured to measure at least 1,5-AG, according to certain embodiments of the present disclosure.

FIG. 6 is a block diagram depicting a computing device configured to perform the operations of FIGS. 4A-5, according to certain embodiments of the present disclosure.

FIGS. 7A-7B depict exemplary enzyme domain configurations for a continuous multi-analyte sensor, according to certain embodiments of the present disclosure.

FIGS. 7C-7D depict exemplary enzyme domain configurations for a continuous multi-analyte sensor, according to certain embodiments of the present disclosure.

FIGS. 8A-8B depict alternative views of an exemplary dual electrode enzyme domain configuration for a continuous multi-analyte sensor, according to certain embodiments of the present disclosure.

FIGS. 8C-8D depict alternative views of an exemplary dual electrode enzyme domain configuration for a continuous multi-analyte sensor, according to certain embodiments of the present disclosure.

FIG. 8E depicts an exemplary dual electrode configuration for a continuous multi-analyte sensor, according to certain embodiments of the present disclosure.

FIGS. 9A-9G depict a single sided, co-planar analyte sensor assembly, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

Accurate assessment of kidney function is important as a screening tool and for monitoring disease progression and guiding prognosis at least with respect to kidney disease, and further with respect to co-morbid diseases and/or conditions that put the patient at higher risk for developing worsening kidney disease. However, existing techniques for diagnosing and staging kidney disease, including albumin-to-creatinine ratio (ACR) tests, glomerular filtration rate (GFR) tests, GFR measurement through insulin clearance tests, and blood tests for monitoring creatinine levels of a patient, face significant challenges with respect to accuracy and reliability. Note that herein, and for purposes of the present disclosure, kidney disease refers to any loss of kidney function, regardless of whether the loss of function would be classified as kidney disease under current scientific standards. Examples of loss of kidney function include a chronic loss of kidney function, referred to as CKD, or an acute loss of kidney function, referred to as AKI, often brought on by sudden injury.

In particular, these existing techniques are imprecise, inefficient, and lack the sensitivity and specificity necessary to accurately diagnose and stage kidney disease prior to a major loss of kidney function. For example, the existing techniques for diagnosing and staging kidney disease are often a single point-in-time reading, which can be influenced by the patient's activity, such as diet or exercise near or during the point in time, or represent a significant biological delay in the patient's kidney function that would not reflect an acute event. In other words, there are currently no therapy management systems with the technical capabilities to measure, process, and analyze one or more analytes in real-time and on a continuous basis to collect enough data over time that allows for detecting kidney disease and providing therapy management therapy management guidance. As a result of these technical problems and deficiencies, monitoring the development of kidney disease at an early stage and/or the progression of kidney disease over time is not technically possible, which, in some cases, might prove to be life threatening for a patient with kidney disease. Therefore, with the existing kidney disease detection techniques, patients are typically not diagnosed with kidney disease until they have lost nearly 50% of their kidney function because, as discussed above, the technologies and techniques available currently only involve the use of point-in-time measurements of certain analytes to assess loss of kidney function. The technical deficiencies associated with existing diagnosis techniques can contribute to a patient experiencing worsening symptoms, an irreversible decline in overall health, and even death given the time-dependent nature of kidney disease.

Accordingly, certain embodiments herein provide a technical solution to the technical problems described above by providing a continuous analyte monitoring system (e.g., including, at least one of a continuous glucose sensor, and/or a continuous 1,5-AG sensor and at least one sensor electronics module) for use in staging, and/or monitoring kidney disease, as well as providing therapy management guidance. Measuring analytes (e.g., glucose and 1,5-AG) in a continuous readout as proposed herein allows for more accurately monitoring patients' kidney function over time, monitoring for the development of early-stage kidney disease, monitoring for the progression of kidney disease over time, and providing real-time therapy management guidance. Such information can also be used to make informed decisions in the assessment of kidney health, treatment of kidney disease, and/or prevention of kidney disease.

Certain embodiments described herein also provide a therapy management system configured to use analyte data generated by the continuous analyte monitoring system described herein to provide kidney disease therapy management guidance. For example, certain embodiments provide methods and systems for continuously monitoring analyte data, for example, one of at least 1,5-AG or glucose levels, and/or non-analyte data to provide kidney disease therapy management guidance, patient-specific feedback (e.g., regarding medical intervention, medication recommendations (e.g., insulin administration), and/or lifestyle recommendations (e.g., maintain a specific exercise regime, etc.)) to prevent the decline, progression and/or development of kidney disease.

By way of background, 1,5-AG, a pyranose sugar, is a naturally occurring monosaccharide. Blood concentrations of 1,5-AG decrease during times of hyperglycemia above 180 mg/dL, and return to normal levels after a relatively short period of time (e.g., across a few days and up to 2 weeks) in the absence of hyperglycemia. As a result, 1,5-AG can be measured, based on which meaningful data and insight can be derived (as described herein) for patients. This can be helpful for patients where traditional measurements are not reliable, such as patients with either type-1 or type-2 diabetes mellitus, when identifying glycemic variability or a history of high blood glucose where current glycemic measurements such as hemoglobin A1c (HbA1c) and blood glucose have near normal values. 1,5-AG is ingested from nearly all foods during the course of a regular diet and is nearly 100% non-metabolized. It is carried in the blood stream and filtered by the glomerulus, where it enters the kidney. Once in the kidney, 1,5-AG is re-absorbed back into the blood through the renal proximal tubule. A small amount, equal to the amount ingested, of 1,5-AG is released in the urine to maintain a constant amount in the blood and tissue.

Glucose, another pyranose sugar, is a competitive inhibitor of 1,5-AG re-absorption in the kidney. If blood glucose levels rise over a certain threshold (referred to herein as “reabsorption threshold”) for any period of time, the kidney cannot re-absorb all of the glucose back into the blood, leading to increased excretion in the urine (glucosuria). As a result, blood levels of 1,5-AG may rapidly respond to the increase in glucose levels and begin to decrease, and continue to decrease until glucose levels fall below the reabsorption threshold. Once the hyperglycemia is corrected, 1,5-AG begins to be re-absorbed from the kidney back into the blood at a steady rate. If an individual's glucose levels remain below the reabsorption threshold for a period of time (e.g., one week and up to 4 weeks), 1,5-AG can return to its normal levels. As a result, measurement of the level of 1,5-AG in the blood is a test for a recent history of hyperglycemic episodes and could also serve as a sensitive and specific indicator of kidney function, including glomerular filtration efficiency. In certain embodiments, a patient's reabsorption threshold is determined, which is the level of the patient's blood glucose where glucose begins to outcompete 1,5-AG for reabsorption and 1,5-AG levels begin to decrease. The reabsorption threshold is directly related to the patient's kidney function and provide further indication of the presence and/or risk of developing kidney disease as described below. The reabsorption threshold can be calculated using the continuous measurement of glucose levels, or can be determined using historical glucose levels for a specific patient, or based on clinical population based values.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insight to be derived using the algorithms described herein for staging and monitoring kidney disease, as well as providing therapy management guidance. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously monitor and stage kidney disease, as well as continuously provide therapy management guidance, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for providing real-time staging and monitoring of kidney disease, as well as real-time therapy management guidance. Real time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose and/or 1,5-AG concentration values using calibration techniques described herein to account for the sensitivity of the continuous analyte sensor(s), and (3) transmitting measured glucose and/or 1,5-AG concentration data, including glucose and/or 1,5-AG concentration values, to a display device via wireless connection.

For example, the at least one sensor electronics module can be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose and/or 1,5-AG concentration data to a display device at a particular transmission period (or rate), which can be the same as (or longer than) the sampling period, such as every 1 minute (0.016 Hz), 5 minutes, 10 minutes, etc.

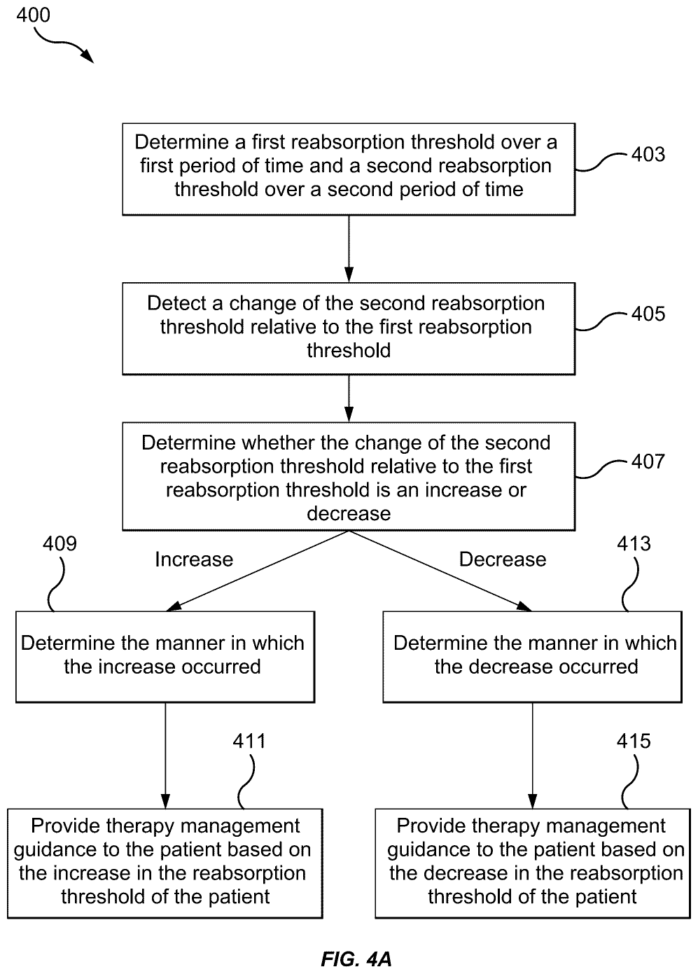

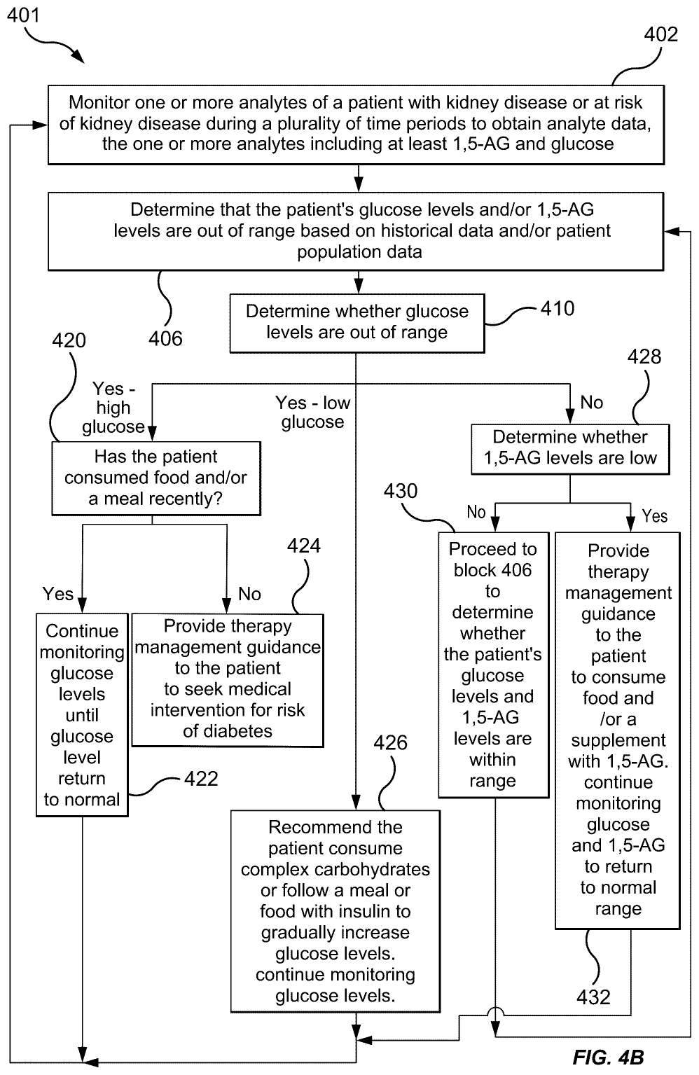

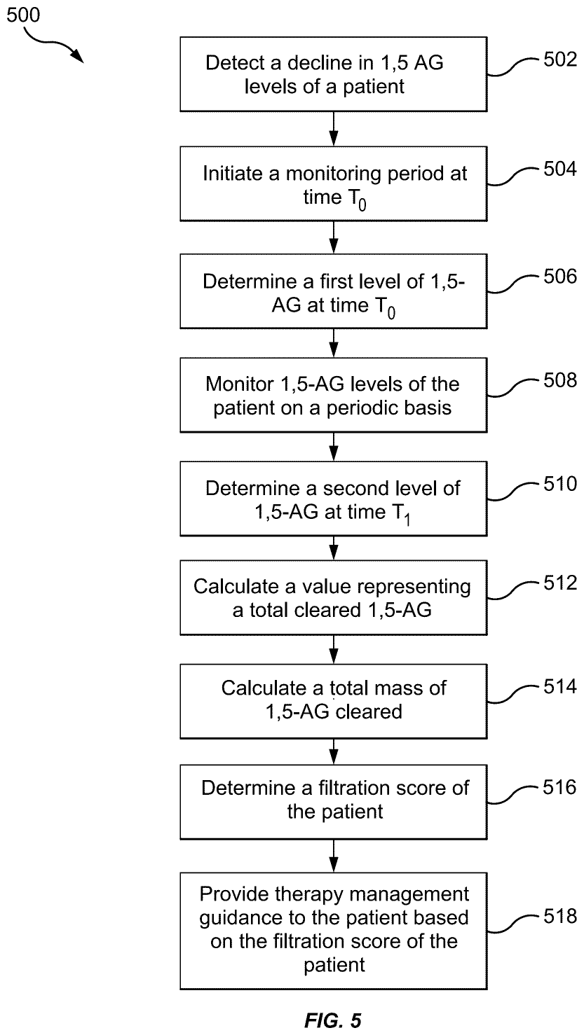

The real-time analyte data that is continuously generated by the continuous analyte monitoring system described herein, therefore, allows the therapy management system herein to monitor and stage kidney disease, as well as provide therapy management guidance, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein for staging and monitoring kidney disease, as well as providing therapy management guidance in a timely manner. In other words, deriving meaningful data and insight from a stream of real-time data that is continuously generated, processed, calibrated, and analyzed, using the algorithms and systems described herein, is not a task that can be mentally performed. For example, executing the algorithm described in relation to FIGS. 4A-5 in real-time and on a continuous basis, which would involve using a stream of real-time data that is continuously generated by a patient's continuous analyte monitoring system and/or significantly large amount of population data (e.g., hundreds or thousands of data points for each one of thousands or millions of patients in the patient population) is not a task that can be mentally performed, especially in real-time at times.

Further, certain embodiments herein are directed to a technical solution to a technical problem associated with analyte sensor systems. In particular, each analyte sensor system that is manufactured by a sensor manufacturer might perform slightly different. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current can be expressed in units of picoA mps (pA) or counts. The amount of measured analyte can be expressed as a concentration level in units of milligrams per deciliter (mg/dL), and the calibration sensitivity can be expressed in units of pA/(mg/dL) or counts/(mg/dL). The calibration baseline refers to the amount of electrical current produced by the analyte sensor when no analyte is detected, and can be expressed in units of pA or counts.

The calibration sensitivity, calibration baseline, and other information related to the sensitivity profile for the analyte sensor system can be programmed into the sensor electronics module of the analyte sensor system during the manufacturing process, and then used to convert the analyte sensor electrical signals into measured analyte concentration levels. For example, the calibration slope (calibration sensitivity) can be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels can be determined using a sensitivity function M(t) that is based on the initial in vivo sensitivity (M₀) and the final in vivo sensitivity (M_{f}). The sensitivity function M(t) can expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$A C L = count / M ( {t}_{i} ) \qquad Eq . 1$$

A calibration baseline (baseline) can also be used to determine a measured analyte concentration level (ACL) from an analyte sensor count value (count) at a time tᵢ, and Equation 2 presents one technique:

$$A C L = ( count - baseline ) / M ( {t}_{i} ) \qquad Eq . 2$$

### Example Therapy Management System Including an Example Analyte Sensor for Determining the Presence, Progression, or Development of Kidney Disease

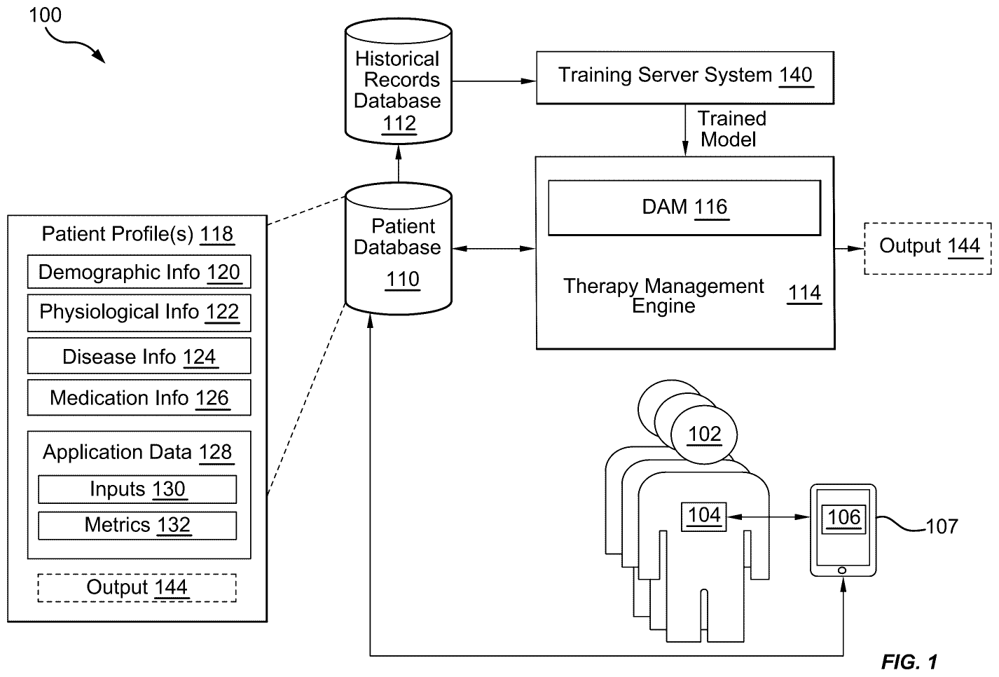

FIG. 1 illustrates an example therapy management system 100 for providing kidney disease therapy management guidance to patients 102 (individually referred to herein as a patient and collectively referred to herein as patients), using a continuous analyte monitoring system 104 configured to continuously measure one or more analytes, such as 1,5-AG and glucose levels. A patient, in certain embodiments, can be a patient with varying stages of kidney disease, a patient with diabetes (and therefore known to be at risk of developing kidney disease), and/or a healthy patient (e.g., a patient not diagnosed with kidney disease and/or diabetes), for example.

In certain embodiments, system 100 includes continuous analyte monitoring system 104, a display device 107 that executes application 106, a therapy management engine 114, a patient database 110, a historical records database 112, a training system 140, and a therapy management engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) or gas (e.g., exhaled air) that can be analyzed. A nalytes can include naturally occurring substances, drugs, artificial substances, metabolites, ions, vitamins, minerals, proteins, enzymes, oligonucleotides, and/or reaction products. A nalytes for measurement by the devices and methods can include, but can not be limited to, potassium, glucose, endogenous insulin, acarboxyprothrombin; acylcarnitine; endogenous insulin; adenine phosphoribosyl transferase; adenosine deaminase; albumin; albumin-creatinine ratio; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-peptide; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; creatinine; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; pyruvate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; proteinuria; quinine; reverse tri-iodothyronine (rT3); selenium; serum pancreatic lipase; sisomicin; somatomedin C; specific antibodies recognizing any one or more of the following that can include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, pro-C3, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include 1,5-AG and glucose, in some cases other analytes listed above can also be considered.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EM R system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system can also be used to create reports for clinical care and/or disease management for a patient. In certain embodiments, the EMR can be in communication with therapy management engine 114 (e.g., via a network) for performing the techniques described herein. In particular, as described herein, therapy management engine 114 can obtain data associated with a patient, use the obtained data as input into one or more trained model(s), and output a prediction. In some cases, the EMR can provide the data to therapy management engine 114 to be used as input into the one or more models. Further, in some cases, therapy management engine 114, after making a prediction, can provide the output prediction to the EMR.

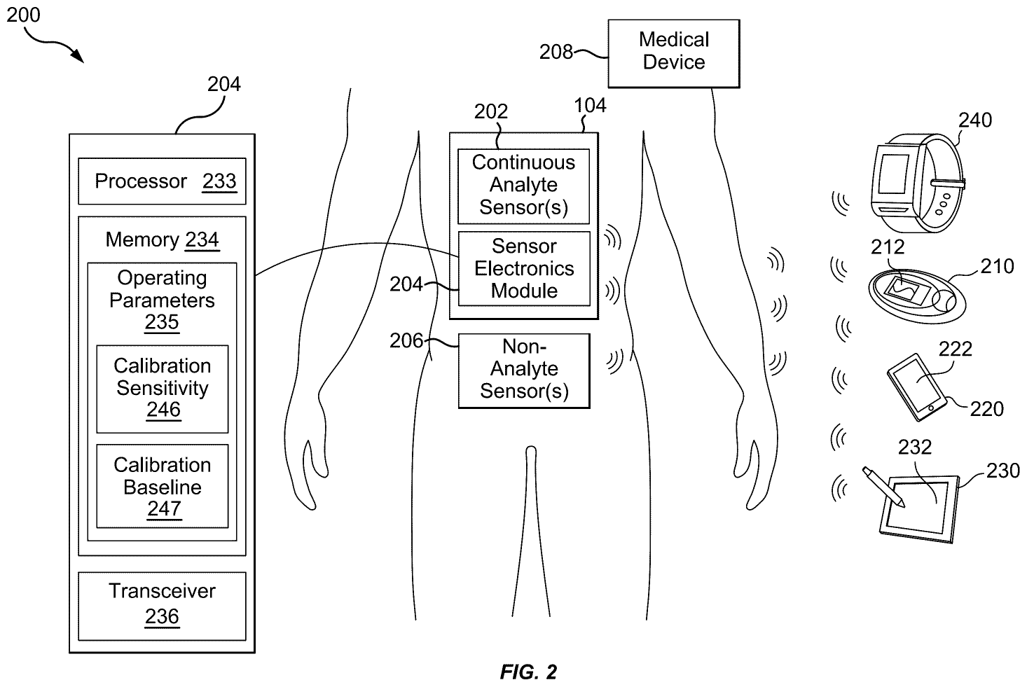

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth (e.g., including Bluetooth Low Energy (BLE)) connection, WiFi connection, local area network connection, cellular network connection, etc.). In certain embodiments, display device 107 is a smart phone. However, in certain other embodiments, display device 107 can instead be any other type of computing device such as a laptop computer, a smart watch, a tablet, a standalone receiver, or any other computing device capable of executing application 106. In some embodiments, continuous analyte monitoring system 104 and/or analyte sensor application 106 transmits the analyte measurements to one or more other individuals having an interest in the health of the patient (e.g., a family member or physician for real-time treatment and care of the patient). Continuous analyte monitoring system 104 is described in more detail with respect to FIG. 2 In certain other embodiments, the continuous analyte monitoring system 104 does not transmit analyte measurements to display device 107 and instead provides the data directly to a third party for diagnostic purposes (e.g., a patient does not have a display device and/or the display device can not be paired with continuous analyte monitoring system).

Application 106 is a mobile health application that is configured to receive and analyze analyte measurements from analyte monitoring system 104. In particular, application 106 stores information about a patient, including the patient's analyte measurements, in a patient profile 118 associated with the patient for processing and analysis, as well as for use by therapy management engine 114 to provide therapy management recommendations or guidance to the patient.

Therapy management engine 114 refers to a set of software instructions with one or more software modules, including data analysis module (DAM) 116. In certain embodiments, therapy management engine 114 executes entirely on one or more computing devices in a private or a public cloud. In such embodiments, application 106 communicates with therapy management engine 114 over a network (e.g., Internet). In some other embodiments, therapy management engine 114 executes partially on one or more local devices, such as display device 107 and/or continuous analyte monitoring system 104, and partially on one or more computing devices in a private or a public cloud. In some other embodiments, therapy management engine 114 executes entirely on one or more local devices, such as display device 107 and/or continuous analyte monitoring system 104. As discussed in more detail herein, therapy management engine 114 can provide therapy management recommendations to the patient via application 106 for medical intervention, medications, and/or lifestyle changes to improve the patient's kidney disease stage, prevent worsening kidney disease, prevent the patient from developing kidney disease, and/or treat kidney disease. Therapy management engine 114 provides therapy management recommendations for medical intervention, medications, and/or lifestyle changes based on information included in patient profile 118.

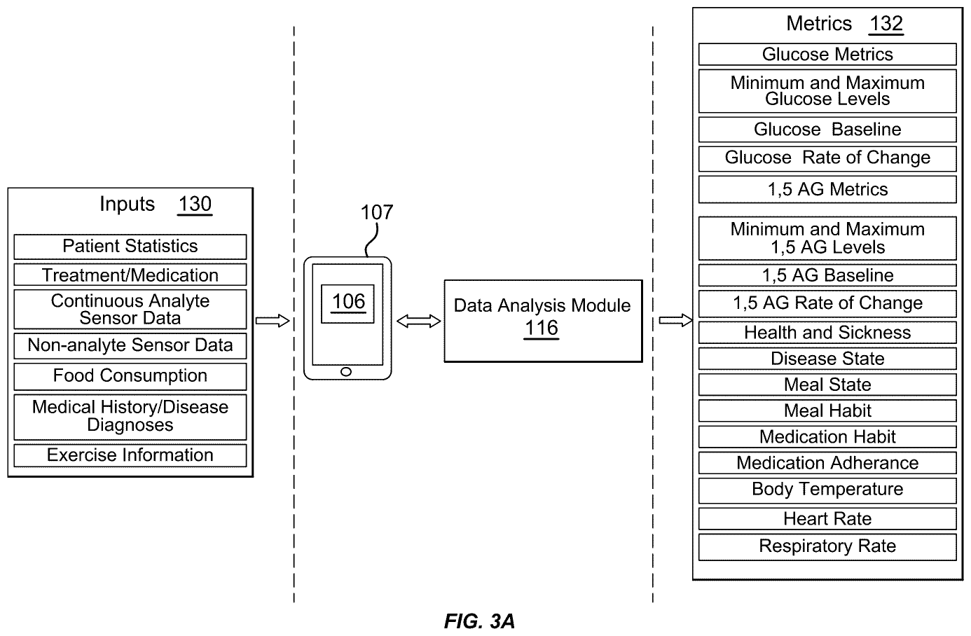

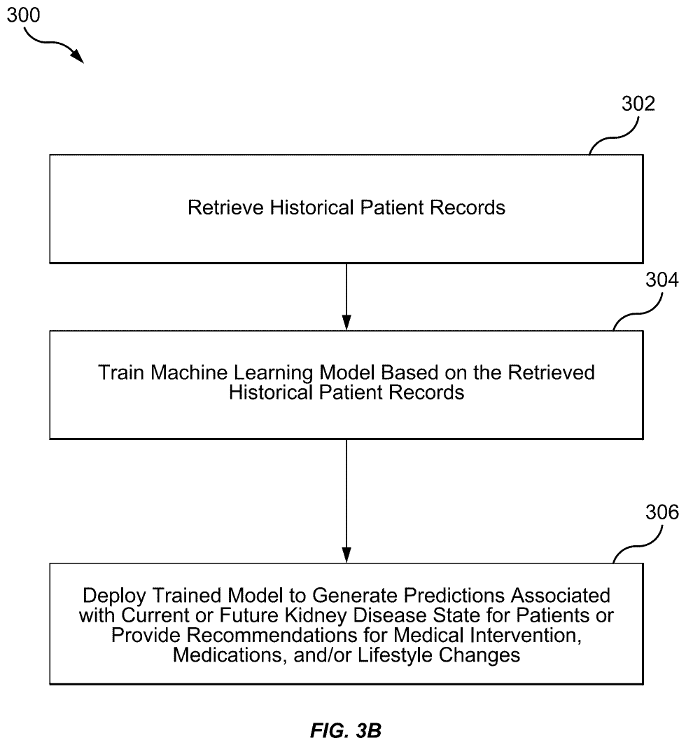

Patient profile 118 can include information collected about the patient from application 106. For example, application 106 provides a set of inputs 130, including the analyte measurements received from continuous analyte monitoring system 104, that are stored in patient profile 118. In certain embodiments, inputs 130 provided by application 106 include other data in addition to analyte measurements received from continuous analyte monitoring system 104. For example, application 106 can obtain additional inputs 130 through manual patient input, one or more other non-analyte sensors or devices, non-continuous analyte lab test results (e.g., kidney biopsy, liver biopsy, metabolic assay panels, Fibroscan results, ultrasound imaging, magnetic resonance imaging, electrolyte panels, urine pH tests, etc.), other applications executing on display device 107, etc. Non-analyte sensors and devices include one or more of, but are not limited to, an insulin pump, stretch sensor, body sound sensor, impedance sensor, an electrocardiogram (ECG) sensor, a heart rate monitor, a blood pressure sensor, a respiratory sensor, a thermometer, sensors or devices provided by display device 107 (e.g., accelerometer, camera, global positioning system (GPS), heart rate monitor, etc.), or other patient accessories (e.g., a smart watch or fitness tracker), or any other sensors or devices that provide relevant information about the patient. Inputs 130 of patient profile 118 provided by application 106 are described in further detail below with respect to FIG. 3A.

DAM 116 of therapy management engine 114 is configured to process the set of inputs 130 to determine one or more metrics 132. Metrics 132, discussed in more detail below with respect to FIG. 3A, can, at least in some cases, be generally indicative of the disease state of a patient, such as one or more of the patient's general analyte trends, trends associated with the health of the patient, etc. In certain embodiments, metrics 132 can then be used by therapy management engine 114 as input for providing kidney disease therapy management guidance to the patient. As shown, metrics 132 are also stored in patient profile 118.

Patient profile 118 also includes demographic info 120, physiological info 122, disease progression info 124, and/or medication info 126. In certain embodiments, such information is provided through patient input, obtained from one or more analyte or non-analyte sensors, or obtained from certain data stores (e.g., electronic medical records (EMRs), etc.). In certain embodiments, demographic info 120 includes one or more of the patient's age, ethnicity, gender, etc. In certain embodiments, physiological info 122 includes one or more of the patient's height, weight, and/or body mass index (BMI). In certain embodiments, disease progression info 124 includes information about a disease of a patient, such as whether the patient has been previously diagnosed with kidney disease, and/or have had symptoms of kidney disease, such as a history of diabetes, liver disease, hypertension, etc. In certain embodiments, information about a patient's disease also includes the length of time since diagnosis, the level of disease control, level of compliance with disease management therapy, predicted kidney function, other types of diagnosis (e.g., heart disease, hypertension, obesity), or measures of health (e.g., heart rate, exercise, sleep, etc.), and/or the like . . .

In certain embodiments, medication info 126 includes information about the amount, frequency, and type of a medication taken by a patient. In certain embodiments, the amount, frequency, and type of a medication taken by a patient is time-stamped and correlated with the patient's analyte levels, thereby, indicating the impact the amount, frequency, and type of the medication had on the patient's analyte levels.

In certain embodiments, medication information includes information about the consumption of one or more drugs known to damage the kidney. One or more drugs known to damage the kidney include nonsteroidal anti-inflammatory drugs (NSAIDS) such as ibuprofen (e.g., Advil, Motrin) and naproxen (e.g., A leve), vancomycin, iodinated radiocontrast (e.g., refers to any contrast dyes used in diagnostic testing), angiotensin-converting enzyme (ACE) such as lisinopril, enalapril, and ramipril, aminoglycoside antibiotics such as neomycdin, gentamicin, tobramycin, and amikacin, antiviral human immunodeficiency virus (HIV) medications, zoledronic acid (e.g., Zometa, Reclast), foscarnet, and the like.

In certain embodiments, medication information includes information about the consumption of one or more drugs known to control the complications of kidney disease. One or more drugs known to control the complications of kidney disease include medications to lower blood pressure and preserve kidney function such as ACE inhibitors or angiotensin II receptor blockers, medications to treat anemia such as supplements of the hormone erythropoietin, medications used to lower cholesterol levels such as statins, medications used to prevent weak bones such as calcium and vitamin D supplements, phosphate binders, and the like.

In certain embodiments, patient profile 118 is dynamic because at least part of the information that is stored in patient profile 118 is revised over time and/or new information is added to patient profile 118 by therapy management engine 114 and/or application 106. Accordingly, information in patient profile 118 stored in patient database 110 provides an up-to-date repository of information related to a patient.

Patient database 110, in some embodiments, refers to a storage server that operates in a public or private cloud. Patient database 110 can be implemented as any type of datastore, such as relational databases, non-relational databases, key-value datastores, file systems including hierarchical file systems, and the like. In some exemplary implementations, patient database 110 is distributed. For example, patient database 110 can comprise a plurality of persistent storage devices, which are distributed. Furthermore, patient database 110 can be replicated so that the storage devices are geographically dispersed.

Patient database 110 includes patient profiles 118 associated with a plurality of patients who similarly interact with application 106 executing on the display devices 107 of the other patients. Patient profiles stored in patient database 110 are accessible to not only application 106, but therapy management engine 114, as well. Patient profiles in patient database 110 are accessible to application 106 and therapy management engine 114 over one or more networks (not shown). As described above, therapy management engine 114, and more specifically DAM 116 of therapy management engine 114, can fetch inputs 130 from patient database 110 and compute a plurality of metrics 132 which can then be stored as application data 128 in patient profile 118.

In certain embodiments, patient profiles 118 stored in patient database 110 are also stored in historical records database 112. Patient profiles 118 stored in historical records database 112 provide a repository of up-to-date information and historical information for each patient of application 106. Thus, historical records database 112 essentially provides all data related to each patient of application 106, where data is stored according to an associated timestamp. The timestamp associated with information stored in historical records database 112 can identify, for example, when information related to a patient has been obtained and/or updated.

Further, historical records database 112 maintains time series data collected for patients over a period of time, including for patients who use continuous analyte monitoring system 104 and application 106. For example, analyte data for a patient who has used continuous analyte monitoring system 104 and application 106 for a period of five years to manage the patient's kidney health has time series analyte data associated with the patient maintained over the five year period.

Further, in certain embodiments, historical records database 112 includes data for one or more patients who are not patients of continuous analyte monitoring system 104 and/or application 106. For example, historical records database 112 includes information (e.g., patient profile(s)) related to one or more patients analyzed by, for example, a healthcare physician (or other known method), and not previously diagnosed with kidney disease, as well as information (e.g., patient profile(s)) related to one or more patients who were analyzed by, for example, a healthcare physician (or other known method) and were previously diagnosed with (varying types and stages of) kidney disease and/or diabetes or other diagnoses known to cause increased risk of kidney disease. Data stored in historical records database 112 is referred to herein as population data, which could include hundreds or thousands of data points for each one of thousands or millions of patients in the patient population. In other words, data stored in historical records database 112 and used in certain embodiments described herein could include gigabytes, terabytes, petabytes, exabytes, etc. of data.

Data related to each patient stored in historical records database 112 provides time series data collected over the disease lifetime of the patient. For example, the data includes information about the patient prior to being diagnosed with kidney disease and/or diabetes and information associated with the patient during the lifetime of the disease, including information related to each stage of the kidney disease and/or diabetes as it progressed and/or regressed in the patient, as well as information related to other diseases, such as hyperkalemia, hypokalemia, diabetes, hypertension, hypotension, cardiac arrythmias, heart conditions and diseases, or similar diseases that are co-morbid in relation to kidney disease. Such information can indicate symptoms of the patient, physiological states of the patient, analyte levels of the patient, states/conditions of one or more organs of the patient, habits of the patient (e.g., activity levels, food consumption, etc.), medication prescribed, etc. throughout the lifetime of the disease.

Although depicted as separate databases for conceptual clarity, in some embodiments, patient database 110 and historical records database 112 operate as a single database. That is, historical and current data related to patients of continuous analyte monitoring system 104 and application 106, as well as historical data related to patients that were not previously patients of continuous analyte monitoring system 104 and application 106, can be stored in a single database. The single database can be a storage server that operates in a public or private cloud.

As mentioned previously, therapy management system 100 is configured to provide kidney disease therapy management to a patient using continuous analyte monitoring system 104. In certain embodiments, therapy management engine 114 is configured to provide real-time and or non-real-time kidney disease therapy management guidance to the patient and or others, including but not limited, to healthcare providers, family members of the patient, caregivers of the patient, researchers, artificial intelligence (AI) engines, and/or other individuals, systems, and/or groups supporting care or learning from the data. In some embodiments, therapy management engine 114 is configured to provide therapy management guidance in the form of alerts, alarms, or notifications to the patient via display device 107. Alerts, alarms, and notifications can be provided in form of tactile, audible, or visual notifications, alarms, or alerts. Notifications, alarms and alerts can be provided to the patient on the display device 107, while application 106 is running, or as background notifications even when application 106 is running in the background. Alarms, alerts, and notifications inform the patient of various therapy management guidance provided by therapy management engine 114, including guidance to seek medical intervention for CKD or AKI, or adjust a medication of the patient. For example, therapy management engine 114 can provide an alert, alarm, and/or notification to the patient to increase or decrease their SGLT2 inhibitor dose.

For example, therapy management engine 114 can be used to collect information associated with a patient in patient profile 118 stored in patient database 110, to perform analytics thereon for providing kidney disease therapy management guidance and, in some cases, for providing recommendations to the patient based on the therapy management guidance. Therapy management engine 114 is also used to collect information associated with a patient in patient profile 118 to perform analytics thereon for providing kidney disease therapy management guidance and providing one or more recommendations for medical intervention, medications, and/or lifestyle changes based, at least in part, on the therapy management guidance. Patient profile 118 is accessible to therapy management engine 114 over one or more networks (not shown) for performing such analytics.

In certain embodiments, therapy management engine 114 further collects information from the patient regarding recent habits in order to determine the accuracy of the kidney disease therapy management guidance. For example, therapy management engine 114 determines, from continuous analyte monitoring system 104, an abnormal pattern of analyte data consistent with worsening disease state and/or reduced kidney function. During time periods of abnormal analyte patterns, therapy management engine 114 reviews information collected from the patient to determine whether the abnormal analyte pattern is a result of worsening disease state or kidney function, or if the abnormal pattern is a result of food consumption, a new medication, or an illness or infection, for example.

Patient profile 118 is accessible to therapy management engine 114 over one or more networks (not shown) for performing such analytics. In certain embodiments, therapy management engine 114 is configured to provide real-time and/or non-real-time therapy management guidance around diabetes to the patient and/or others, including but not limited, to healthcare providers (HCP), family members of the patient, caregivers of the patient, researchers, and/or other individuals, systems, and/or groups supporting care or learning from the data.

In certain embodiments, therapy management engine 114 utilizes one or more trained machine learning models capable of providing kidney disease therapy management guidance based on information that therapy management engine 114 has collected/received from patient profile 118. In the illustrated embodiment of FIG. 1 therapy management engine 114 utilizes trained machine learning model(s) provided by a training system 140. Although depicted as a separate server for conceptual clarity, in certain embodiments, training system 140 and therapy management engine 114 operates as a single server or system. That is, the model is trained and used by a single server, or is trained by one or more servers and deployed for use on one or more other servers or systems. In certain embodiments, the model is trained on one or many virtual machines (VMs) running, at least partially, on one or many physical services in relational and or non-relational database formats.

Training system 140 is configured to train the machine learning model(s) using training data, which includes data (e.g., from patient profiles) associated one or more patients (e.g., patients or non-patients of continuous analyte monitoring system 104 and/or application 106) previously diagnosed with varying stages of kidney disease, previously diagnosed with varying stages of diabetes at risk of developing kidney disease, as well as patients not previously diagnosed with kidney disease and/or diabetes (e.g., healthy patients, etc.). The training data is stored in historical records database 112 and is accessible to training system 140 over one or more networks (not shown) for training the machine learning model(s).

The training data refers to a dataset that has been featurized and labeled. For example, the dataset includes a plurality of data records, each including information corresponding to a different patient profile stored in patient database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a patient, which is reflected in a corresponding data record, is a feature used in training the machine learning model. Such features include demographic information (e.g., age, gender, ethnicity, etc.), analyte information (e.g., 1,5-AG metrics, glucose metrics, etc.), non-analyte sensor information (e.g., stretch sensor data, impedance sensor data, body sound sensor data, etc.), medical history and/or disease information (e.g., kidney disease (e.g., CKD, kidney failure, acquired cystic kidney disease, kidney stones, multicystic dysplastic kidney, nephrotic syndrome, polycystic kidney disease (PKD)), liver disease (e.g., cirrhosis), diabetes, blood pressure measurements, albumin-to-creatinine ratio (ACR) tests, glomerular filtration rate (GFR) tests, blood tests for monitoring potassium levels historical patient kidney metabolic panels, etc.), medication information, and/or any other information relevant to providing kidney disease diagnosis and stage predictions, or to providing recommendations to patients.

In addition, the data record is labeled with information the corresponding model is being trained to predict. In one example, if a model is being trained to predict whether the patient's kidney disease is worsening, then the data records in the training dataset are labeled with such diagnoses. In another example, if a model is being trained to output a prediction related to kidney function and/or kidney disease development, then the data records in the training dataset are labeled with one or more of such diagnoses. Note that, in one example, such a model is a multi-input single-output (M ISO) model, configured to predict only whether the patient's kidney disease is worsening, in which case additional MISO models are trained to each predict the patient's kidney health and/or risk of developing kidney disease, including whether the condition is getting better or worsening, or the like. In another example, such a model is a multi-input multi-output (MIMO) model, configured to predict multiple disease-related predictions (e.g., kidney disease progression, kidney function, kidney disease development, and/or kidney disease risk predictions, etc.).

The model(s) are then trained by training system 140 using the featurized and labeled training data. In particular, the features of each data record are used as input into the machine learning model(s), and the generated output is compared to label(s) associated with the corresponding data record. The model(s) computes a loss based on the difference between the generated output and the provided label(s). This loss is then used to modify the internal parameters or weights of the model. By iteratively processing each data record corresponding to each historical patient, the model(s) is iteratively refined to generate accurate predictions of a kidney disease progression, kidney function, kidney disease development, or recommendations for medical intervention, medications, and/or lifestyle changes, etc.

As illustrated in FIG. 1 training system 140 deploys these trained model(s) to therapy management engine 114 for use during runtime. Training system 140 can include one or more computer systems, each including one or more servers or one or more other types of computing devices or systems. For example, therapy management engine 114 obtains patient profile 118 associated with a patient and stored in patient database 110, use information in patient profile 118 as input into the trained model(s), and output a prediction indicative of the patient's kidney disease progression, kidney function, kidney disease development, and/or feedback related to kidney disease (e.g., shown as output 144 in FIG. 1). Output 144 generated by therapy management engine 114 indicates improvement or deterioration in the patient's kidney disease over time. Output 144 is provided to the patient (e.g., through application 106), to a caretaker of the patient (e.g., a parent, a relative, a guardian, a teacher, a physical therapist, a fitness trainer, a nurse, etc.), to a physician or healthcare provider of the patient, or any other individual that has an interest in the wellbeing of the patient for purposes of improving the health of the patient, such as, in some cases by effectuating recommended treatment and/or seeking medical intervention. Output 144 generated by therapy management engine 114 is stored in patient database 110 and is utilized to train or re-train the trained model(s) and/or update a rules-based model.

In certain embodiments, output 144 generated by therapy management engine 114 is stored in patient profile 118. Output 144 can be indicative of a patient's current or future kidney disease state, kidney function, and recommendations for medical intervention, medications, lifestyle changes, etc. Output 144 stored in patient profile 118 can be continuously updated by therapy management engine 114. Accordingly, for example, disease states and recommendations, originally stored as outputs 144 in patient profile 118 in patient database 110 and then passed to historical records database 112, provide an indication of the progression or improvement of the disease state of a patient over time, as well as provide an indication as to the effectiveness of different medical intervention, medications, and lifestyle changes recommended to the patient to improve disease state.

In certain embodiments, a patient's own historical data is used by training system 140 to train a personalized model for the patient that provides therapy management guidance and insight around the patient's medical history/current disease state, average analyte levels, etc. For example, in certain embodiments, a model trained based on population data is used to provide disease progression feedback to the patient. However, after collecting personalized information (e.g., analyte sensor information, non-analyte sensor information, disease state, etc.) associated with the patient, the personalized information is used to further personalize the model. For example, information obtained over time from the patient is used to more accurately determine kidney disease development and/or progression, provide personalized recommendations for medical intervention, medications, and/or lifestyle changes, and monitor regression of disease state over time.

Further, a patient's historical data can be used to generate a baseline to indicate progression or regression in the patient's kidney disease based, for example, on the patient's analyte metrics (e.g., baseline, rate of change, minimum and/or maximum levels), etc. As an illustrative example, a patient's data, including a plurality of analyte measurements, over the course of 2 weeks during a previous time period (e.g., 1 day, 1 week, 1 month) can be used to generate a baseline that can be compared with the patient's current data to identify whether the patient's kidney disease has improved. In certain embodiments, the model is further able to predict or project out the patient's kidney disease and its future improvement/deterioration based on the patient's recent pattern of data (e.g., analyte data, non-analyte data, meal trends, exercise trends, etc.).

In certain embodiments, historical patient population data based on patients with kidney disease and/or diabetes is used to generate a baseline to indicate progression or regression in the patient's kidney disease. In certain other embodiments, known clinical evidence and/or observable data through clinical investigations of procedures can be used to generate a baseline to indicate progression or regression in the patient's kidney disease.

In certain embodiments, an AI/ML model is trained to provide a recommendation for medical intervention, medication, lifestyle, and other types of therapy management recommendations to help the patient improve their kidney disease state based on the patient's historical data, including how different types of medication, food, and/or activities impacted the patient's kidney function in the past. In certain embodiments, an AI/ML model is trained to predict the underlying cause of certain improvements or deteriorations in the patient's kidney disease state and/or risk of developing kidney disease. For example, application 106 displays a user interface with a graph that shows the patient's analyte levels with trend lines and indicate, e.g., retrospectively, how the body's analyte levels affected the state of the patient's kidney disease at certain points in time.

In certain other embodiments, rules-based models are used. For example, a rules-based model is used to map a patient's inputs, analyte or non-analyte data from one or more continuous analyte sensor(s) 202 and/or non-analyte sensor(s) 206, and/or historical data to certain current or future kidney disease state, risk of developing kidney disease, recommendations for medical intervention, medications, lifestyle changes, etc., using, for example, a rules library. In certain embodiments, a rules-based model maps certain inputs to kidney disease state predictions, a certain risk of developing kidney disease, and/or recommendations for patients based on patients with similar inputs in the past. Some example rules are discussed herein in relation to methods 400 and 401.

FIG. 2 is a diagram 200 conceptually illustrating an example continuous analyte monitoring system 104 including example continuous analyte sensor(s) with sensor electronics, in accordance with certain aspects of the present disclosure. For example, continuous analyte monitoring system 104 is configured to continuously monitor one or more analytes of a patient, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 can be in wireless communication (e.g., directly or indirectly) with one or more of display devices 210, 220, 230, and 240. In certain embodiments, sensor electronics module 204 can also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 can comprise one or more sensors for detecting and/or measuring analyte(s). The continuous analyte sensor 202 can be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 can be configured to continuously measure analyte levels of a patient using one or more techniques, such as enzymatic techniques, chemical techniques, physical techniques, electrochemical techniques, potentiostatic techniques, potentiometric techniques, impedimetric techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes of the patient. The data stream can include raw data signals, which are then converted into a calibrated and/or filtered data stream used to provide estimated analyte value(s) to the patient.

In certain embodiments, the continuous analyte sensor 202 can be a multi-analyte sensor, configured to continuously measure multiple analytes in a patient's body. For example, in certain embodiments, the continuous multi-analyte sensor 202 can be a single sensor configured to measure glucose and/or 1,5-AG in the patient's body.

In certain embodiments, one or more multi-analyte sensors can be used in combination with one or more single analyte sensors. Information from each of the multi-analyte sensor(s) and single analyte sensor(s) can be combined to provide therapy management guidance using methods described herein. In further embodiments, other non-contact and or periodic or semi-continuous, but temporally limited, measurements for physiological information can be integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 202 can comprise a percutaneous wire that has a proximal portion coupled to the sensor electronics module 204 and a distal portion with several electrodes, such as a measurement electrode and a reference electrode. The measurement (or working) electrode can be coated, covered, treated, embedded, etc., with one or more chemical molecules that react with a particular analyte, and the reference electrode can provide a reference electrical voltage. The measurement electrode can generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis.

In certain embodiments, the continuous analyte sensor(s) 202 can comprise a planar substrate that has a proximal portion coupled to the sensor electronics module 204 and a distal portion with several electrodes such as a measurement electrode and a reference electrode. The measurement (or working) electrode can be coated, covered, treated, embedded, etc., with one or more chemical molecules that react with a particular analyte, and the reference electrode can provide a reference electrical voltage. The measurement electrode can generate the analog electrical signal, which is conveyed along a conductor that extends from the measurement electrode to the proximal portion of the continuous analyte sensor(s) 202 that is coupled to the sensor electronics module 204. After the continuous analyte monitoring system 104 has been applied to epidermis of the host, continuous analyte sensor(s) 202 penetrates the epidermis, and the distal portion extends into the dermis and/or subcutaneous tissue under epidermis.

In certain embodiments, in addition to the planar and coaxial sensors described herein, other configurations of the planar and coaxial continuous analyte sensor(s) 202 can also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 can include a single-analyte sensor configured to measure glucose concentration levels, and another single-analyte sensor configured to measure 1,5-AG concentration levels of the patient. As another illustrative example, continuous analyte sensor(s) 202 can include a single-analyte sensor configured to measure glucose concentration levels, and one or more multi-analyte sensors configured to measure 1,5-AG concentration levels, potassium concentration levels, lactate concentration levels, creatinine concentration levels, etc. As yet another illustrative example, continuous analyte sensor(s) 202 can include a multi-analyte sensor configured to measure glucose concentration levels, 1,5-AG concentration levels, potassium concentration levels, lactate concentration levels, creatinine concentration levels, etc. Accordingly, continuous analyte sensor(s) 202 is configured to generate at least one analog electrical signal that is proportional to the concentration level of a particular analyte, and sensor electronics module 204 is configured to convert the analog electrical signal into an analyte sensor count values, calibrate the analyte sensor count values based on the sensitivity profile of the continuous analyte sensor(s) 202 to generate measured analyte concentration levels, and transmit the measured analyte concentration level data, including the measured analyte concentration levels, to a display device, such as display devices 210, 220, 230, and/or 240, via a wireless connection. For example, sensor electronics module 204 can be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which can be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 can incorporate a thermocouple within, or alongside, the percutaneous wire to provide an analog temperature signal to the sensor electronics module 204, which can be used to correct the analog electrical signal or the measured analyte data for temperature. In other embodiments, the thermocouple can be incorporated into the sensor electronics module 204 above the adhesive pad, or, alternatively, the thermocouple can contact the epidermis of the patient through openings in the adhesive pad.

In certain embodiments, the sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 can be a general-purpose or application-specific microprocessor, an application-specific integrated circuit (A SIC), a field programmable gate array (FPGA), etc., that executes instructions to perform control, computation, input/output, etc, functions for the sensor electronics module 204. Processor 233 can include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry can be combined into a system-on-chip (SoC).

Generally, processor 233 can be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, and generate measured analyte data from the measured analyte concentration levels, generate sensor data packages that include, inter alia, the measured analyte concentration level data. Processor 233 can store the measured analyte concentration level data in memory 234, and generate the sensor data packages at regular intervals (such as the transmission period) for transmission by wireless transceiver 236 to a display device, such as display devices 210, 220, 230, and/or 240. Processor 233 can also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

In various embodiments, memory 234 can include volatile and nonvolatile medium. For example, memory 234 can include combinations of random access memory (RAM), dynamic RAM (DRAM), static RAM (SRAM), read only memory (ROM), flash memory, cache memory, and/or any other type of non-transitory computer-readable medium. Memory 234 can store one or more analyte sensor system applications, modules, instruction sets, etc. for execution by processor 233, such as instructions to generate measured analyte data from the analyte sensor count values, etc.

Memory 234 can also store certain sensor operating parameters 235, such as a calibration slope (or calibration sensitivity), a calibration baseline, etc. In particular, the calibration sensitivity, calibration baseline, and other information related to the sensitivity profile for the sensor electronics module 204 can be programmed into the sensor electronics module 204 during the manufacturing process, and then used to convert the analyte sensor electrical signals into measured analyte concentration levels. For example, as discussed above, the calibration slope can be used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (M_{CC}) 246 and/or calibration baseline 247 can be stored in memory 234.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) or releasably attachable to continuous analyte sensor(s) 202. Sensor electronics module 204 can include hardware, firmware, and/or software that enable measurement of levels of analyte(s) via continuous analyte sensor(s) 202. For example, sensor electronics module 204 can include an electrochemical analog front end (e.g., a potentiostat, galvanostat, coulostat, etc.), a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (A SIC), a microcontroller, and/or a processor.

Display devices 210, 220, 230, and/or 240 are configured for displaying displayable sensor data, including analyte data, which can be transmitted by sensor electronics module 204. Each of display devices 210, 220, 230, or 240 can include a display such as a touchscreen display 212, 222, 232, and/or 242 for displaying sensor data to a patient and/or for receiving inputs from the patient. For example, a graphical user interface (GUI) can be presented to the patient for such purposes. In certain embodiments, the display devices can include other types of user interfaces such as a voice user interface instead of, or in addition to, a touchscreen display for communicating sensor data to the patient of the display device and/or for receiving patient inputs. Display devices 210, 220, 230, and 240 can be examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices can include a custom display device specially designed for displaying certain types of displayable sensor data associated with analyte data received from sensor electronics module. In certain embodiments, the plurality of display devices can be configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end patient) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 can be in communication with a medical device 208. Medical device 208 can be a passive device in some example embodiments of the disclosure. For example, medical device 208 can be an insulin pump for administering insulin to a patient. For a variety of reasons, it can be desirable for such an insulin pump to receive and track glucose values and/or 1,5-AG values transmitted from continuous analyte monitoring systems 104, where continuous analyte sensor 202 is configured to measure at least glucose and 1,5-AG.

Further, as mentioned, sensor electronics module 204 can also be in communication with other non-analyte sensors 206. Non-analyte sensors 206 can include, but are not limited to, an altimeter sensor, an accelerometer sensor, a global positioning system (GPS) sensor, a temperature sensor, a respiration rate sensor, a stretch sensor, an impedance sensor, a body sound sensor, electrophysiological sensors, opto-physiological sensors, etc. Non-analyte sensors 206 can also include monitors such as heart rate monitors, blood pressure monitors, pulse oximeters, caloric intake monitors, indirect calorimetry devices and medicament delivery devices. One or more of these non-analyte sensors 206 can provide data to therapy management engine 114 described further below. In some aspects, a patient can manually provide some of the data for processing by training system 140 and/or therapy management engine 114 of FIG. 1

In certain embodiments, non-analyte sensors 206 can further include sensors for measuring skin temperature, core temperature, sweat rate, and/or sweat composition.

In certain embodiments, the non-analyte sensors 206 can be combined in any other configuration, such as, for example, combined with one or more continuous analyte sensors 202. As an illustrative example, a non-analyte sensor, e.g., a body sounds sensor, can be combined with a continuous glucose and/or 1,5-AG sensor 202 to form a glucose, 1,5-AG, and body sounds sensor used to transmit sensor data to the sensor electronics module 204 using common communication circuitry.

In certain embodiments, a wireless access point (WAP) can be used to couple one or more of continuous analyte monitoring system 104, the plurality of display devices, medical device(s) 208, and/or non-analyte sensor(s) 206 to one another. For example, WAP can provide Wi-Fi and/or cellular (e.g., 4G, LTE, 5G, 6G, LTE CAT-M1, NB-IoT, WiMAX, UWB) connectivity among these devices. Near Field Communication (NFC), Thread home automation communication system, Matter home automation communication system, and/or Bluetooth can also be used among devices depicted in diagram 200 of FIG. 2

FIG. 3A illustrates example inputs and example metrics that are calculated based on the inputs for use by the therapy management system of FIG. 1, according to some embodiments disclosed herein. In particular, FIG. 3A provides a more detailed illustration of example inputs and example metrics introduced in FIG. 1.

FIG. 3A illustrates example inputs 130 on the left, application 106 and DAM 116 in the middle, and metrics 132 on the right. In certain embodiments, each one of metrics 132 can correspond to one or more values, e.g., discrete numerical values, ranges, or qualitative values (high/medium/low, stable/unstable, etc.). Application 106 obtains inputs 130 through one or more channels (e.g., manual patient input, sensors, other applications executing on display device 107, an EMR system, etc.). As mentioned previously, in certain embodiments, inputs 130 can be processed by DAM 116 to output a plurality of metrics, such as metrics 132. Inputs 130 and metrics 132 can be used by training system 140 and therapy management engine 114 to both train and deploy one or more machine learning models for providing kidney disease therapy management guidance to the patient, and other functionalities described herein.

In certain embodiments, starting with inputs 130, patient statistics, such as one or more of age, gender, height, weight, BMI, body composition (e.g., % body fat), stature, build, or other information can also be provided as an input. In certain embodiments, patient statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which can, for example, communicate with the display device 107 to provide patient data.

In certain embodiments, treatment/medication information is also provided as an input. Medication information can include information about the type, dosage, and/or timing of when one or more medications are to be taken by the patient. As mentioned herein, the medication information can include information about one or more glycemic controlling medications (e.g., Metformin), glucagon-like peptide-1 receptor agonists (GLP-1) medications, one or more drugs known to damage the kidney, one or more drugs known to control the complications of kidney disease that are prescribed to the patient, and/or one or more medications for treating one or more symptoms of kidney disease, hyperkalemia, hypokalemia, diabetes, and/or other conditions and diseases the patient can have. The input related to medication information can be one or more medication and/or pill trackers to monitor oral medication consumption. Treatment information can include information regarding different lifestyle habits, surgical procedures, and/or other non-invasive procedures recommended by the patient's physician. For example, the patient's physician can recommend a patient increase/decrease their glucose intake, exercise for a minimum of thirty minutes a day, and/or increase an insulin dosage or other medication to maintain, and/or improve, kidney health, glucose homeostasis, general health, etc. In certain embodiments, treatment/medication information can be provided through manual patient input.

In certain embodiments, analyte sensor data can also be provided as input, for example, through continuous analyte monitoring system 104. In certain embodiments, analyte sensor data can include 1,5-AG and/or glucose levels measured by at least a single analyte sensor (or multi-analyte sensor) in continuous analyte monitoring system 104.

In certain embodiments, input can also be received from one or more non-analyte sensors, such as non-analyte sensors 206 described with respect to FIG. 2 Input from such non-analyte sensors 206 can include information related to a heart rate, a heart rate variability, a respiration rate, a respiration rate variability, oxygen saturation, blood pressure, or a body temperature (e.g. to detect illness, physical activity, etc.) of a patient. In certain embodiments, electromagnetic sensors can also detect low-power radio frequency (RF) fields emitted from objects or tools touching or near the object, which can provide information about patient activity or location.

In certain embodiments, input received from non-analyte sensors can include input relating to a patient's insulin delivery. In particular, input related to the patient's insulin delivery can be received, via a wireless connection on a smart insulin pen, via patient input, and/or from an insulin pump. Insulin delivery information can include one or more of insulin volume, time of delivery, etc. Other parameters, such as insulin action time, insulin activity rate or duration of insulin action, can also be received as inputs.

In certain embodiments, input received from non-analyte sensors can include input relating to a patient's medication delivery (e.g., through injectable drug injectors and/or pumps). Example medications can include those that affect metabolic function and/or analyte levels (such as glucose and 1,5-AG). For example, these medications can include GLP-1 medications, SGLT2 inhibitor medications, metformin or other glucose reducing medications, and other similar medications with similar effects. In particular, input related to the patient's medication delivery can be received, via a wireless connection on a drug delivery injector and/or pump and/or via patient input. Medication delivery information can include one or more of GLP-1 dose, time of delivery, dose frequency, mode of delivery, etc.

In certain embodiments, starting with inputs 130, food consumption information can include information about one or more of meals, snacks, and/or beverages, such as one or more of the size, content (carbohydrate, fat, protein, etc.), sequence of consumption, and time of consumption. In certain embodiments, food consumption can be provided by a patient through manual entry, by providing a photograph through an application that is configured to recognize food types and quantities, and/or by scanning a bar code or menu. In various examples, meal size can be manually entered as one or more of calories, quantity (“three cookies”), menu items (“Royale with Cheese”), and/or food exchanges (1 fruit, 1 dairy). In some examples, meal information can be received via a convenient user interface provided by application 106.

In certain embodiments, food consumption information (the type of food (e.g., liquid or solid, snack or meal, etc.) and/or the composition of the food (e.g., carbohydrate, fat, protein, etc.)) can be determined automatically based on information provided by one or more sensors. Some example sensors can include body sound sensors (e.g., abdominal sounds can be used to detect the types of meal, e.g., liquid/solid food, snack/meal, etc.), radio-frequency sensors (e.g., read relevant nutritional contact from an RFID IC embedded in the packaging of a food or beverage item), cameras, hyperspectral cameras, and/or analyte (e.g., glucose, creatinine, lactate, etc.) sensors to determine the type and/or composition of the food.

In certain embodiments, medical history and/or disease diagnoses (e.g., kidney disease, hyperkalemia, hypokalemia, diabetes, hypertension, heart conditions and diseases, liver disease, blood pressure measurements, albumin-to-creatinine ratio (ACR) tests, glomerular filtration rate (GFR) tests, blood tests for monitoring potassium or creatinine levels, historical patient kidney metabolic panels etc.) can be provided as an input. For example, the patient can have an existing diagnosis of kidney disease and this diagnosis can be provided through manual patient input. In certain embodiments, disease diagnoses can also be provided by interfacing with an electronic source such as an electronic medical record.

In certain embodiments, time can also be provided as an input, such as time of day or time from a real-time clock. For example, in certain embodiments, input analyte data can be timestamped to indicate a date and time when the analyte measurement was taken for the patient.

Patient input of any of the above-mentioned inputs 130 can be provided through continuous analyte monitoring system 104, non-analyte sensors 206, and/or a user interface, such a user interface of display device 107 of FIG. 1 As described above, in certain embodiments, DAM 116 determines or computes the patient's metrics 132 based on inputs 130. An example list of metrics 132 is shown in FIG. 3A.

In certain embodiments, glucose metrics can be determined from sensor data (e.g., glucose measurements obtained from a continuous glucose sensor of continuous analyte monitoring system 104). For example, glucose metrics refer to time-stamped glucose measurements or values that are continuously generated and stored over time. In some examples, glucose metrics can also be determined, for example, based upon historical data in particular situations, e.g., given a combination of food consumption, insulin, and/or exercise.

In certain embodiments, a minimum and maximum glucose level can be determined from sensor data. For example, daily minimum and maximum glucose values for each day over a specified amount of time (e.g., a week or a month) can be determined. In certain embodiments, the minimum and maximum glucose levels can be determined based on an average minimum and maximum over a specified amount of time (e.g., a week or a month). In certain embodiments, DAM 116 can continuously or periodically calculate a normal glucose range and time-stamp and store the corresponding information in the patient's profile 118.

In other embodiments, a normal minimum and maximum glucose level can be determined from population data (e.g., from data records or historical patients with kidney disease). In such embodiments, each patient can have personalized, customized, acceptable glucose minimum and/or maximum glucose values, which can be determined based on time periods when the patient is in a fasting state or during a meal, for example.

In certain embodiments, a glucose baseline can be determined from sensor data (e.g., glucose measurements obtained from a continuous glucose sensor of continuous analyte monitoring system 104). A glucose baseline represents a patient's normal glucose levels during periods where fluctuations in glucose production is typically not expected. A patient's baseline glucose level is generally expected to remain constant over time, unless challenged through an action such as consuming food or exercise by the patient, for example. Additionally, a patient's baseline glucose level can also change based on the patient's health, specifically an improvement or decline in liver health and/or kidney health. Further, each patient can have a different glucose baseline. In certain embodiments, a patient's glucose baseline can be determined by calculating an average of glucose levels over a specified amount of time where fluctuations are not expected.

For example, the baseline glucose level for a patient can be determined over a period of time when the patient is sleeping, sitting in a chair, or other periods of time where the patient is sedentary and not consuming food or medication which would reduce or increase glucose levels. In certain embodiments, DAM 116 can continuously, semi-continuously, or periodically calculate a glucose baseline and time-stamp and store the corresponding information in the patient's profile 118. In certain embodiments, DAM 116 can calculate the glucose baseline using glucose levels measured over a period of time where the patient is sedentary, the patient is not consuming glucose-heavy foods, and where no external conditions exist that would affect the glucose baseline.

In certain other embodiments, DAM 116 can use glucose levels measured over a period of time where the patient is, at least for a subset of the period of time, engaging in exercise and/or consuming glucose and/or an external condition exists that would affect the glucose baseline level. In this case, in some examples, DAM 116 can first identify which measured glucose values are to be used for calculating the baseline glucose level by identifying glucose values that can have been affected by an external event, such the consumption of food, exercise, medication, or other perturbation that would disrupt the capture of a glucose baseline measurement. DAM 116 can then exclude such measurements when calculating the glucose baseline level of the patient. In some other examples, DAM 116 can calculate the glucose baseline level by first determining a percentage of the number of glucose values measured during a specific time period that represent the lowest glucose values measured. DAM 116 can then take an average of this percentage to determine the glucose baseline level.

In certain embodiments, a glucose rate of change can be determined from glucose levels (e.g., glucose measurements obtained from a continuous glucose sensor of continuous analyte monitoring system 104). A glucose rate of change refers to a rate that indicates how one or more time-stamped glucose measurements or values change in relation to one or more other time-stamped glucose measurements or values. Glucose rates of change can be determined over one or more seconds, minutes, hours, days, etc. Further, glucose rate of change can be positive, negative, or an absolute value.

In certain embodiments, a reabsorption threshold can be determined. The reabsorption threshold refers to the glucose level at which glucose outcompetes 1,5-AG for absorption and therefore causes 1,5-AG to be cleared (filtered) rather than reabsorbed through the kidney to the bloodstream. 1,5-AG is normally reabsorbed in the kidneys, meaning it is taken back into the bloodstream from the filtered fluid. When glucose outcompetes 1,5-AG for absorption, it means that elevated blood glucose levels are exceeding the kidney's capacity to reabsorb 1,5-AG, which results in a decrease in serum 1,5-AG levels. Generally, for a healthy patient, the clinical reabsorption threshold where 1,5-AG starts to clear rather than be reabsorbed is at or around 180 mg/dL. However, the reabsorption threshold can be patient specific and differ depending on both the glycemic health and kidney health of the patient. Furthermore, the reabsorption threshold can differ across different populations based on age, gender, or based on specific health conditions.

As such, as described in further detail below, the embodiments herein allow for determining a patient specific reabsorption threshold by continuously monitoring glucose and 1,5-AG levels over time and identifying the glucose level at which 1,5-AG levels begin to fall. In certain embodiments, the reabsorption threshold can represent a running average of a plurality of reabsorption thresholds calculated for a patient over time. For example, a first reabsorption threshold, a second reabsorption threshold, and a third reabsorption threshold can be calculated for the patient over a first time period, and the average of the three reabsorption thresholds can be used as the patient's reabsorption rate to provide a more accurate representation of the patient's reabsorption threshold typical and to mitigate the effect of outlier or anomalous readings.

In certain embodiments, 1,5-AG metrics can be determined from sensor data (e.g., 1,5-AG measurements obtained from a continuous 1,5-AG sensor of continuous analyte monitoring system 104). For example, 1,5-AG metrics refer to time-stamped 1,5-AG measurements or values that are continuously generated and stored over time.

In certain embodiments, a minimum and maximum 1,5-AG level can be determined from sensor data. For example, a minimum and maximum 1,5-AG values for each day over a specified amount of time (e.g., a week or a month) can be determined. In certain embodiments, the minimum and maximum 1,5-AG levels can be determined based on an average minimum and maximum over a specified amount of time (e.g., a week or a month). In certain embodiments, DAM 116 can continuously or periodically calculate a normal 1,5-AG range and time-stamp and store the corresponding information in the patient's profile 118.

In other embodiments, a normal minimum and maximum 1,5-AG level can be determined from population data (e.g., from data records or historical patients with kidney disease). In such embodiments, each patient can have personalized, customized, acceptable minimum and/or maximum 1,5-AG values, which can be determined based on various time periods when the patient is in a fasting state or during a meal, for example.

In certain embodiments, a 1,5-AG baseline can be determined from sensor data (e.g., 1,5-AG measurements obtained from a continuous 1,5-AG sensor of continuous analyte monitoring system 104). The 1,5-AG baseline represents a patient's normal 1,5-AG levels during periods where fluctuations in 1,5-AG production is typically not expected. A patient's baseline 1,5-AG level is generally expected to remain constant over time, unless challenged through an action such as consuming food or a supplement that is high in 1,5-AG, for example. Additionally, a 1,5-AG baseline can be determined during periods of time when the patient is not experiencing high glucose levels. High glucose levels can cause a lower 1,5-AG baseline, and therefore, can be an indicator that 1,5-AG is being outcompeted for absorption.

Additionally, a patient's baseline 1,5-AG level can also change based on the patient's health, specifically an improvement or decline in kidney health, for example. Further, each patient can have a different 1,5-AG baseline. In certain embodiments, a patient's 1,5-AG baseline can be determined by calculating an average of 1,5-AG levels over a specified amount of time where fluctuations are not expected.

For example, the baseline 1,5-AG level for a patient can be determined over a period of time when the patient is sleeping, sitting in a chair, or other periods of time where the patient is sedentary and not consuming food or medication which would reduce or increase 1,5-AG levels. In certain embodiments, DAM 116 can continuously, semi-continuously, or periodically calculate a 1,5-AG baseline and time-stamp and store the corresponding information in the patient's profile 118. In certain embodiments, DAM 116 can calculate the 1,5-AG baseline using 1,5-AG levels measured over a period of time where the patient is sedentary, the patient is not consuming high 1,5-AG foods, and where no external conditions exist that would affect the 1,5-AG baseline. In certain embodiments, DAM 116 can calculate the 1,5-AG baseline level by first determining a percentage of the number of 1,5-AG values measured during a specific time period that represent the lowest 1,5-AG values measured. DAM 116 can then take an average of this percentage to determine the 1,5-AG baseline level.

In certain embodiments, a 1,5-AG rate of change can be determined from 1,5-AG levels (e.g., 1,5-AG measurements obtained from a continuous 1,5-AG sensor of continuous analyte monitoring system 104). The 1,5-AG rate of change refers to a rate that indicates how one or more time-stamped 1,5-AG measurements or values change in relation to one or more other time-stamped 1,5-AG measurements or values. 1,5-AG rates of change can be determined over one or more seconds, minutes, hours, days, etc. Further, 1,5-AG rate of change can be positive, negative, or an absolute value. Note that a 1,5-AG rate of change can indicate a 1,5-AG rate of change of absorption or a 1,5-AG rate of change of clearance, which are different metrics. For example, if the measured rate of change is positive, then the rate of change indicates a rate of change of absorption, while a negative rate of change could indicate a rate of change of clearance. The 1,5-AG rate of change of clearance at various glucose levels, assuming 1,5-AG production is constant, is directly correlated to kidney health.

In certain embodiments, one or more glucose metrics and/or 1,5-AG metrics can be determined over one or more periods of time after the consumption of food containing 1,5 AG (e.g., meat, soybeans etc.), or 1-5AG supplements. In such an example, the therapy management engine first determines whether the patient has reached a maximum 1-5AG baseline level for the patient. As described, 1-5AG is a stable analyte, for which a maximum 1-5AG level occurs over a period of time and can be determined at the peak of the 1-5AG curve once there is no more increase in 1-5AG. This maximum is usually the normal range of 1-5AG in the patient's body when the kidney's are reabsorbing 1-5AG for a period of time (which usually occurs, if glucose values do not outcompete the 1-5AG for enough time). Once the therapy management engine has determined that the patient is at their baseline (maximum) 1-5AG level, the engine can determine the patient's glucose level and monitor for an increase at or above an absorption threshold for the patient. This can be achievable by monitoring for when a patient has a spike in glucose level (or a gradual increase), which can occur in response to foods, or therapy management engine 114 can instruct the patient to consume glucose in an OGTT. Therapy management engine 114 can then monitor the glucose level at which 1,5-AG levels begin to decrease to determine the patient's reabsorption threshold.

In certain embodiments, health and sickness metrics can be determined, for example, based on one or more of patient input (e.g., pregnancy information or known sickness and/or infection information), from physiologic sensors (e.g., temperature), activity sensors, or a combination thereof. In certain embodiments, based on the values of the health and sickness metrics, for example, a patient's state can be defined as being one or more of healthy, ill, rested, or exhausted.

In certain embodiments, disease stage metrics, such as for kidney disease, can be determined, for example, based on one or more of patient input or output provided by therapy management engine 114 illustrated in FIG. 1. In certain embodiments, example disease stages for kidney disease, can include AKI, stage 1 CKD with normal or high GFR (e.g., GFR>90 mL/min), stage 2 mild CKD (e.g., GFR=60-89 mL/min), stage 3A moderate CKD (e.g., GFR=45-59 mL/min), stage 3B moderate CKD (e.g., GFR=30-44 mL/min), stage 4 severe CKD (e.g., GFR=15-29 mL/min), and stage 5 end stage CKD (e.g., GFR<15 mL/min). In certain embodiments, example disease stages can be represented as a GFR value/range, severity score, and the like.

In certain embodiments, the meal state metric can indicate the state the patient is in with respect to food consumption. For example, the meal state can indicate whether the patient is in one of a fasting state, pre-meal state, eating state, post-meal response state, or stable state. In certain embodiments, the meal state can also indicate nourishment on board, e.g., meals, snacks, or beverages consumed, and can be determined, for example from food consumption information, time of meal information, and/or digestive rate information, which can be correlated to food type, quantity, and/or sequence (e.g., which food/beverage was eaten first.).

In certain embodiments, meal habits metrics are based on the content and the timing of a patient's meals. For example, if a meal habit metric is on a scale of 0 to 1, the better/healthier the meal consumed by the patient, the higher the meal habit metric of the patient will be to 1, in an example. Better/healthier meals can be defined as those that do not drive analyte (e.g., glucose) levels of a patient out of a normal range for the patient (e.g., 70-180 mg/dl glucose or the patient's desired range). Also, the more the patient's food consumption adheres to a certain time schedule, the closer their meal habit metric will be to 1, in the example. In certain embodiments, the meal habit metrics can reflect the contents of a patient's meals where, e.g., three numbers can indicate the percentages of carbohydrates, proteins and fats.

In certain embodiments, medication habit metrics are based on the patient's prescribed medications and a determination of whether the prescribed medications can have an effect on the patient's analyte levels. For example, by analyzing a patient's medication habits, DAM 116 can determine whether the patient's medications can impact the patient's analyte measurements at a particular time. For example, if the patient is taking an SGLT2 inhibitor, the SGLT2 inhibitor can block reabsorption of 1,5-AG. In that case, a decrease in the patient's 1,5-AG levels is directly correlated to the amount of 1,5-AG filtered out of the patient's kidney, and therefore, the kidney health of the patient.

Based on the patient's medication habits, DAM 116 can determine whether the patient's analyte levels are a result of medication consumption or worsening kidney function, for example. Medication habit metrics can be time-stamped so that they can be correlated with the patient's analyte levels at the same time.

In certain embodiments, based on the patient's medication habits, therapy management engine 114 can determine a clearance rate of 1,5-AG for the patient. Specifically, because SGLT2 inhibitors accelerates 1-5AG filtration, by decreasing the glucose threshold at which 1-5AG is outcompeted, the 1,5-AG must be filtered through the patient's kidney and excreted through urine. The presence of radiolabeled 1,5-AG in the patient's urine is directly indicative of the clearance rate of 1,5-AG through the patient's kidney. For example, if the patient is taking an SGLT2 inhibitor, the patient can be instructed to consume a radiolabeled 1,5-AG supplement, and subsequently provide a urine sample, to monitor the clearance of the radiolabeled 1,5-AG through the patient's kidney. Alternatively, as described below, the 1-5AG rate of clearance measurement without the use of a radiolabeled supplement can also be used to determine the patient's kidney health.

Alternatively, therapy management engine 114 can instruct a patient, who is not taking a SGLT2 inhibitor, to consume a radiolabeled 1,5-AG supplement to determine a 1,5-AG absorption rate and a 1,5-AG clearance rate. In such an example, the patient is also re-absorbing 1,5-AG and, therefore, as the patient's urine is monitored for the presence of radiolabeled 1,5-AG to determine the 1,5-AG clearance rate, the 1,5-AG absorption rate can also be inferred based on the 1,5-AG clearance rate and the 1,5-AG levels of the patient. The 1,5-AG clearance rate and absorption rate can be further monitored based on known glucose levels of the patient during specified time periods to determine how various glucose levels compete with 1,5-AG for reabsorption as described herein.

In certain embodiments, medication adherence is measured by one or more metrics that are indicative of how committed the patient is towards their medication regimen. In certain embodiments, medication adherence metrics are calculated based on one or more of the timing of when the patient takes medication (e.g., whether the patient is on time or on schedule), the type of medication (e.g., is the patient taking the right type of medication), and the dosage of the medication (e.g., is the patient taking the right dosage).

In certain embodiments, body temperature metrics can be calculated by DAM 116 based on inputs 130, and more specifically, non-analyte sensor data from a temperature sensor. In certain embodiments, heart rate metrics can be calculated by DAM 116 based on inputs 130, and more specifically, non-analyte sensor data from a heart rate sensor. In certain embodiments, respiratory rate metrics can be calculated by DAM 116 based on inputs 130, and more specifically, non-analyte sensor data from a respiratory rate sensor.

In certain embodiments, machine learning models deployed by therapy management engine 114 include one or more models trained by training system 140, as illustrated in FIG. 1 FIG. 3A describes in further detail techniques for training the machine learning model(s) deployed by therapy management engine 114 for predicting a current or future kidney disease state and/or providing recommendations for medical intervention, medications, and/or lifestyle changes.

FIG. 3B is a flow diagram depicting a method 300 for training machine learning models to classify a patient, predict a patient's current or future kidney disease state and/or provide recommendations to a patient based on disease state. In certain embodiments, the method 300 is used to train models for predicting a current or future kidney disease state, as illustrated in FIG. 1

Method 300 begins, at block 302, by training server system, such as training system 140 illustrated in FIG. 1 retrieving data from historical records database, such as historical records database 112 illustrated in FIG. 1 As mentioned herein, historical records database 112 can provide a repository of up-to-date information and historical information for patients of a continuous analyte monitoring system and connected mobile health application, such as patients of continuous analyte monitoring system 104 and application 106 illustrated in FIG. 1 as well as data for one or more patients who are not, or were not previously, patients of continuous analyte monitoring system 104 and/or application 106. In certain embodiments, historical records database 112 can include one or more data sets of historical patients who are healthy patients, patients with kidney disease, and/or patients with diabetes.

Retrieval of data from historical records database 112 by training system 140, at block 302, can include the retrieval of all, or any subset of, information maintained by historical records database 112. For example, where historical records database 112 stores information for 100,000 patients (e.g., non-patients and patients of continuous analyte monitoring system 104 and application 106), data retrieved by training system 140 to train one or more machine learning models can include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (A Pls), Health Level 7 (HL7), and or other computer interface language can enable aggregation of healthcare historical records for baseline assessment in addition to the aggregation of de-identifiable patient data from a cloud based repository. Similarly, when integrating into the medical record databases, the integration can be accomplished by directly interfacing with the electronic medical record system or through one or more intermediary systems (e.g., an interface engine, etc.).

As an illustrative example, at block 302, training system 140 can retrieve information for 100,000 patients with various disease states (e.g., healthy patient, patient with kidney disease, and/or a patient with diabetes) stored in historical records database 112 to train a model to predict a current or future kidney disease state of a patient and provide recommendations to the patient. Each of the 100,000 patients can have a corresponding data record (e.g., based on their corresponding patient profile), stored in historical records database 112. Each patient profile 118 can include information, such as information discussed with respect to FIG. 3A.

The training system 140 then uses information in each of the records to train an artificial intelligence or ML model (for simplicity referred to as “ML model” herein). Examples of types of information included in a patient's patient profile were provided above. The information in each of these records can be featurized (e.g., manually or by training system 140), resulting in features that can be used as input features for training the ML model. For example, a patient record can include or be used to generate features related to the patient's demographic information (e.g., an age of a patient, a gender of the patient, etc.), analyte information, such as 1,5-AG and glucose metrics, non-analyte information, and/or any other data points in the patient record (e.g., inputs 130, metrics 132, etc.). Features used to train the machine learning model(s) can vary in different embodiments.

In certain embodiments, each historical patient record retrieved from historical records database 112 is further associated with a label indicating a medical diagnosis of the patient (e.g., a healthy patient, a patient with kidney disease, and/or a patient with diabetes), current kidney disease state, etc. What the record is labeled with would depend on what the model is being trained to predict.

At block 304, method 300 continues by training system 140 training one or more machine learning models based on the features and labels associated with the historical patient records. In some embodiments, the training server does so by providing the features as input into a model. This model can be a new model initialized with random weights and parameters, or can be partially or fully pre-trained (e.g., based on prior training rounds). Based on the input features, the model-in-training generates some output. In certain embodiments, the output can include a current or future kidney disease state and/or recommendations for medical intervention, medications, and/or lifestyle changes to improve the patient's kidney disease state, or similar outputs. Note that the output could be in the form of a classification, a recommendation, and/or other types of output.

In certain embodiments, training system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict a current or future kidney disease state, and/or provide recommendations for treatment, medications, and/or lifestyle changes to improve the patient's kidney disease state more accurately.

One of a variety of machine learning algorithms can be used for training the model(s) described above. For example, one of a supervised learning algorithm, a neural network algorithm, a deep neural network algorithm, a deep learning algorithm, etc. can be used.

At block 306, training system 140 deploys the trained model(s) to make predictions associated with current or future kidney disease state during runtime. In some embodiments, this includes transmitting some indication of the trained model(s) (e.g., a weights vector) that can be used to instantiate the model(s) on another device. For example, training system 140 can transmit the weights of the trained model(s) to therapy management engine 114, which could execute on display device 107, etc. The model(s) can then be used to determine, in real-time, a current or future kidney disease state of a patient using application 106, and/or make other types of recommendations discussed above. In certain embodiments, the training system 140 can continue to train the model(s) in an “online” manner by using input features and labels associated with new patient records.

Further, similar methods for training illustrated in FIG. 3A using historical patient records can also be used to train models using patient-specific records to create more personalized models for making predictions associated with a current or future kidney disease state. For example, a model trained using historical patient records that is deployed for a particular patient, can be further re-trained after deployment. For example, the model can be re-trained after the model is deployed for a specific patient to create a more personalized model for the patient. The more personalized model can be able to more accurately make predictions on disease state of the patient based on the patient's own data (as opposed to only historical patient record data), including the patient's own inputs 130 and metrics 132.

### Example Methods for Providing Kidney Therapy Management Guidance to a Patient Using Continuously Monitored Analyte Data

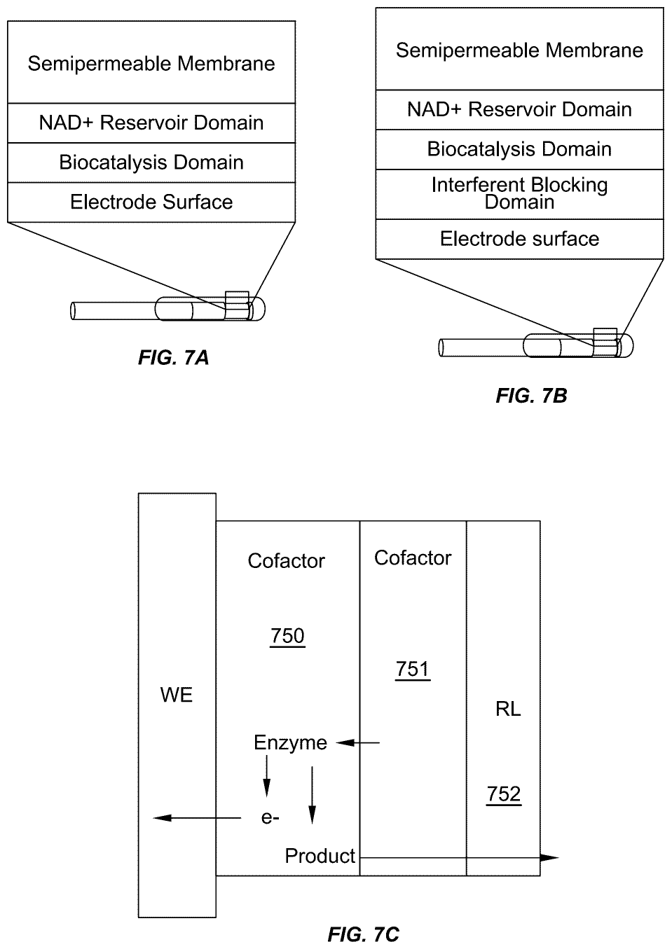

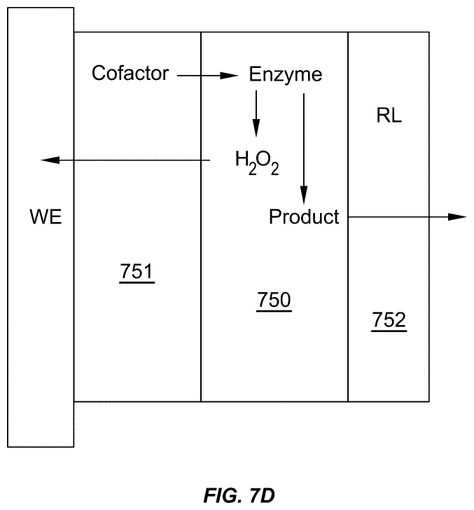

FIG. 4A is a flow diagram illustrating example method 400 for providing kidney disease therapy management guidance using a continuous analyte monitoring system including, at least, a continuous sensor to monitor glucose and 1,5-AG levels, in accordance with certain embodiments described herein. For example, the method is performed to provide kidney disease therapy management guidance to a patient based on one or more analyte levels including at least glucose and 1,5-AG levels. FIG. 4B is a flow diagram illustrating an example method 401 for providing guidance to a patient to ensure that the patient's analyte levels are within range in order to then calculate a reabsorption threshold for the patient, as described in reference to FIGS. 4A and 5A. Methods 400 and 401 are described below with reference to FIGS. 1-3 and their components. As described in methods 400 and 401, FIGS. 7A-9G and their components may be utilized to continuously monitor analyte data.

Method 400 is performed by therapy management engine 114 to collect and/or generate data such as inputs 130 and metrics 132, including, for example, analyte data, patient information, and non-analyte sensor data, as mentioned above, to provide therapy management guidance related to the presence or stage of kidney disease, and/or recommendations to improve kidney function and kidney disease stage. For example, therapy management engine 114 can perform method 400 by monitoring one or more analytes of a patient during a plurality of time periods, the one or more analytes including at least 1,5-AG. In some examples, both glucose and 1,5-AG are monitored in parallel. In some examples, glucose is measured at a trigger point in time, for example, at or near a time where 1,5-AG levels begin to decline, determined using a change from the 1,5-AG baseline threshold being passed and/or detected a downward rate of change of 1,5-AG, indicating the threshold glucose level where glucose begins to outcompete 1,5-AG has been reached, to develop a threshold glucose level. In yet some other examples, glucose may not be measured and a reabsorption threshold may be assigned to the patient based on historical glucose information for the patient or based on patient demographics based on population data. Therapy management engine 114 then determines one or more glucose metrics and 1,5-AG metrics for the patient, and generate a prediction associated with kidney disease stage based on, at least, the glucose metrics and 1,5-AG metrics. In certain embodiments, therapy management engine 114 provides recommendations for treatment (e.g., seek medical intervention) based on the kidney disease prediction.

As discussed above, therapy management engine 114 uses one of a variety of models to provide kidney disease therapy management guidance to the patient, and/or provide recommendations to the patient to seek medical intervention, medications, and/or lifestyle changes based on the inputs. As described, the inputs include analyte data (e.g., received by continuous analyte monitoring system 104), non-analyte data, and/or other patient information (e.g., retrieved from the patient's profile or received via patient inputs). In embodiments where a rules-based model is used, the inputs of the model are mapped to certain kidney disease therapy management guidance, for example. For example, the rules-based model takes inputs and determines whether the patient is at risk of developing kidney disease, has kidney disease, or is suffering from worsening kidney disease.

As an example rule, therapy management engine 114 can determine a patient has worsening kidney disease based on inputs indicating that the patient is diagnosed with mild kidney disease and additional analyte data from continuous analyte monitoring system 104 demonstrating the patient's reabsorption threshold has changed, either increased or decreased, from a first reabsorption threshold over time. In another example, therapy management engine 114 can determine a patient is developing kidney disease and/or experiencing worsening kidney disease or kidney function, or worsening glucose management, based on current and/or historic 1,5-AG data demonstrating the patient's 1,5-AG baseline level decreases over time, in the absence of medication use, such as SGLT2 inhibitors. In another example, therapy management engine 114 can determine a patient is at risk of developing kidney disease based on current and/or historic analyte data demonstrating a change in the reabsorption threshold over time, but above a threshold indicative of mild kidney disease. In another example, therapy management engine 114 can determine a patient has worsening or improving kidney disease. In such an example, therapy management engine 114 can provide a recommendation to the patient to seek medical intervention for worsening kidney disease based on current and/or historic 1,5-AG and glucose data demonstrating the patient's reabsorption threshold is decreasing over time to a level indicative of a loss of kidney function. By monitoring the reabsorption threshold, therapy management engine 114 can determine a patient is experiencing kidney or glucose issues. The reabsorption threshold, the rate at which the change in reabsorption threshold occurs (e.g., over hours, days or longer), the level of the reabsorption threshold baseline, 1,5-AG baseline, or other glucose or 1,5-AG metrics can be used by the therapy management engine 114 to determine whether the change in reabsorption threshold is an indication of the patient developing CKD or progressing to later stages of CKD, or the patient developing AKI. Similarly, the change in the reabsorption threshold at which 1,5-AG begins to clear rather than be reabsorbed can indicate an improvement in kidney function.

In certain embodiments, the rules become more granular, such that a combination of rules and/or inputs allow therapy management engine 114 to output a prediction of kidney disease and/or diabetes, for example.

In certain embodiments, instead of a rules-based model, an AI/ML model is used to output a prediction of development of kidney disease, worsening kidney disease, and/or recommendations for treatment, medications, and/or lifestyle changes, for example. Some or all of the inputs are used as input into the model that is trained to output a disease prediction, disease stage, and/or recommendations for treatment, medications, and/or lifestyle changes for a patient. In such cases, the model is trained using a dataset, including historical population-based data of many patients, who have already been determined to be at risk of developing kidney disease, have worsening kidney disease, various stages of kidney disease (e.g., mild, moderate, and/or severe), and/or diabetes. In such an example, the training dataset is labeled with such determinations.

These rules are used to detect a change in the kidney function of the patient by determining whether the change in reabsorption threshold is increasing or decreasing over time. The rules can further be used to determine the root cause of the change in the reabsorption threshold and/or the manner in which the change occurred. For example, an increase in the reabsorption threshold for a patient is an indication of early stage chronic kidney function failure (e.g., early stage CKD), or of worsening diabetes. Alternatively, a decrease in the reabsorption threshold is an indication of progression to later stages of chronic kidney function failure (e.g., later stages of CKD) or an acute loss of kidney function (e.g., AKI).

The method 400 begins at block 403, when therapy management engine 114 determines a first reabsorption threshold for a patient at a first point in time, or over a first time period, and a second reabsorption threshold for the patient at a second point in time, or over a second time period.

In some embodiments, the first and second reabsorption thresholds are generated by continuously monitoring glucose levels and 1,5-AG levels for the patient. For example, the therapy management engine 114 can first determine the first reabsorption threshold (e.g., the glucose level at which 1,5-AG levels are at a baseline level (i.e., at a relative maximum)) for a patient. The determination of the first reabsorption threshold can be done as an average over several time periods (e.g., time periods during the first time period) where 1,5-AG levels are stable (e.g., where 1,5-AG is being reabsorbed by the kidney in the blood stream and the glucose levels are such that they do not outcompete 1,5-AG for reabsorption).

In another example, the first reabsorption level can be determined using historical glucose or 1,5-AG levels for a patient. In yet another example, the first reabsorption threshold can be determined based on population data for a group of patients that are similar to the patient based on patient profile 118 (e.g., demographic information 120, physiological information 122, disease information 124, medication information 126, or other determinative factors).

In one example, the first reabsorption threshold can be set at a static value. In another example, the first reabsorption threshold is a cumulative average of the patient's reabsorption thresholds that is periodically updated as glucose levels and 1,5-AG levels are monitored. In one example, the first reabsorption threshold is a calculated reabsorption threshold based on monitored glucose levels and 1,5-AG levels for the patient, and the calculated reabsorption threshold is further adjusted based on information within the patient profile 118.

Once therapy management engine 114 determines the first reabsorption threshold based on one or more of the examples described above, therapy management engine 114 can determine the second reabsorption threshold. Therapy management engine 114 determines the second reabsorption threshold by monitoring 1,5-AG and determining when 1,5-AG begins to decline (e.g., a baseline threshold of 1,5-AG is passed or a rate of change of 1,5-AG begins to decline). At the time 1,5-AG begins to decline, the glucose level of the patient is then measured (or estimated) at or near the time corresponding to the initiation of the decline in 1,5-AG. This determination of the second reabsorption threshold can be a single measurement of the reabsorption threshold, or an average, maximum, minimum, or median of reabsorption thresholds of the patient over a period of time.

Following block 403, at block 405, therapy management engine 114 determines whether a change of the second reabsorption threshold of the patient relative to the first reabsorption threshold of the patient can be detected. In other words, therapy management engine 114 compares the second reabsorption threshold of the patient with the first reabsorption threshold of the patient to determine the change between the first and second reabsorption thresholds of the patient. In certain embodiments, the change can be compared against an expected variation in reabsorption thresholds to determine if the change exceeds the expected variation. For example, if the patient's first reabsorption threshold is 180 mg/dL, the expected variation can be 5 mg/dL. Thus, if the patient's second reabsorption threshold is 176 mg/dL, therapy management engine 114 does not detect a change. Alternatively, if the patient's second reabsorption threshold is 170 mg/dL, therapy management engine 114 detects a change. If therapy management engine 114 detects a change, the method 400 continues to block 407. If not, therapy management engine 114 may return to block 403 to continue monitoring the first and second reabsorption threshold.

At block 407, a determination is made whether the change of the second reabsorption threshold relative to the first reabsorption threshold is an increase or a decrease. That is, whether the second reabsorption threshold for the current period of time or point in time (i.e., the most current reabsorption threshold) is higher or lower than the first reabsorption threshold for the patient. If an increase is detected, the method 400 continues to block 409. If, on the other hand, a decrease is detected, the method 400 continues to block 413.

At block 409, in response to therapy management engine 114 determining that the change in reabsorption threshold indicates an increase in the reabsorption threshold, therapy management engine 114 determines the manner in which the increase occurred. For example, an increase in reabsorption threshold is generally an indication of worsening glycemic control and early stages of chronic kidney failure or kidney function decline.

However, if the increase in the reabsorption threshold indicates a return from a previous decrease in the reabsorption threshold or a return from a previous reabsorption threshold that was below an expected threshold (e.g., lower than the healthy reabsorption threshold of 180 mg/dL), the increase indicates an improvement in a condition that previously caused the reabsorption threshold of the patient to decrease to an undesirable level. Such conditions include later stages or worsening of chronic kidney function failure (e.g., as a result of advanced CKD) or an acute worsening of kidney function (e.g., as a result of AKI). The detection of these conditions is discussed in further detail below.

Following block 409, at block 411, therapy management engine 114 provides therapy management guidance to the patient based on the detected increase in the patient's reabsorption threshold. If the increase in the patient's reabsorption threshold indicates a return from a previous decrease in the reabsorption threshold or a reabsorption threshold that was below an expected threshold, therapy management engine 114 can continue monitoring the reabsorption threshold over time. If the increase in reabsorption threshold is not a return from a previous decrease, therapy management engine 114 provides therapy management guidance to the patient to seek medical intervention for early stage chronic kidney failure and/or a decline in kidney health.

At block 413, in response to therapy management engine 114 determining that the change in reabsorption threshold indicates a decrease in the reabsorption threshold, therapy management engine 114 determines the manner in which the decrease occurred. This determination can based on one or more of a magnitude of change of the reabsorption threshold over the time period between the first and second reabsorption thresholds, the time it took for the change to occur, and whether a change over several periods of time has been detected.

There can be several reasons why a decrease in the reabsorption threshold may occur. The first is a chronic worsening of kidney function, which occurs, for example, as a patient enters later stages of chronic kidney failure (e.g., later stages of CKD). In this case, the decrease in the reabsorption threshold for the patient would be gradual and would happen over a longer period of time (e.g., weeks, months, or years). Thus, if therapy management engine 114 determines a gradual decrease in reabsorption threshold over weeks, months, or years, therapy management engine 114 determines the patient has or is developing late stage chronic kidney failure.

Alternatively, a relatively sudden change in the reabsorption threshold (e.g., over hours or days, rather than weeks, months, or years), typically indicates an acute loss of kidney function, which can indicate AKI. Thus, if the change happens suddenly, therapy management engine 114 determines the patient is experiencing AKI. Therapy management engine 114 can continue monitoring the patient's reabsorption threshold to determine if the patient's reabsorption threshold continues to decrease indicating a further decline in kidney function over time. If the reabsorption threshold continues to decrease over time following the sudden decrease, therapy management engine 114 determines that the patient has developed chronic kidney failure as a result of the AKI.

Alternatively, where a decrease is observed, in the rate of clearance of 1-5AG, if the rate of change is occurring at a slower pace but is also observed at a lower reabsorption threshold, then the patient may be experiencing kidney damage to the proximal tubial. This would also indicate AKI. In another example, the determination of root cause as CKD rather than AKI may be based on prior patient history, that is if the patient is in later stages of CKD, the engine may determine that the patient is in final stages of CKD, with the more rapid change of 1-5AG reabsorption threshold.

Conversely, as therapy management engine 114 continues to monitor the patient's reabsorption threshold, the reabsorption threshold may increase over time following the decrease. In one example, this may be a gradual increase over time, for example in response to food consumption, the increase can also occur in response to the patient taking a 1-5AG supplement. In this example, the rate at which the increase in the reabsorption threshold occurs can also be determined. Therapy management engine 114 can then use the increase and the rate at which the increase in the reabsorption threshold occurred to determine if the AKI has been reversed (e.g., an increase is detected in the reabsorption threshold to a healthy reabsorption threshold of 180 mg/dL) and the time it takes for the reversal. This pattern of increase in the reabsorption threshold, including rate of increase, can also be used to titrate medication used to assist in the reversal of AKI.

Thus, if the decrease in the reabsorption threshold follows a previous increase in the reabsorption threshold from the first reabsorption threshold, or if the decrease is towards a more acceptable reabsorption threshold than the patient previously experienced (i.e., a decrease in the reabsorption threshold that causes the reabsorption threshold to come closer to the healthy reabsorption threshold of 180 mg/dL), then an improvement in the patient's kidney function is detected.

Following block 413, at block 415, therapy management engine 114 may provide therapy management guidance to the patient based on the detected decrease in the patient's reabsorption threshold. Therapy management engine 114 may provide therapy management guidance to the patient that their kidney function has improved if the decrease in the reabsorption threshold follows a previous increase in the reabsorption threshold (e.g., from the first reabsorption threshold), or if the decrease is towards the healthy reabsorption threshold (e.g., 180 mg/dL).

Alternatively, if the patient experiences a gradual decline in reabsorption threshold over weeks, months, or years, therapy management engine 114 may provide therapy management guidance to the patient to seek medical intervention for CKD. If the patient experiences a sudden decline in reabsorption threshold over hours or days, therapy management engine 114 may provide therapy management guidance to the patient to seek medical intervention for AKI.

In certain embodiments, a patient is prescribed a medication that affects their reabsorption threshold, such as an SGLT2 inhibitor. If a patient is known to be on an SGLT2 inhibitor, therapy management engine 114 determines the patient's reabsorption threshold when the patient is not on an SGLT2 inhibitor medication and compares the reabsorption threshold of the patient prior to SGLT2 inhibitor medication to the reabsorption threshold after the patient begins taking an SGLT2 inhibitor medication. If the time period between when the patient is and is not taking an SGLT2 inhibitor medication is sequential or otherwise close in time (e.g., a few days or weeks apart), a lower reabsorption threshold when the patient is on an SGLT2 inhibitor medication can be attributed to the SGLT2 inhibitor medication. In this case, therapy management engine 114 determines the decrease in reabsorption threshold is not due to improving kidney health (e.g., following a previous increase in reabsorption threshold), or due to the development of AKI or CKD as described in reference to block 413. This change in reabsorption threshold as a result of SGLT2 inhibitor medication can be used as individual and/or population data to determine an expected decrease in reabsorption threshold as a result of SGLT2 inhibitor medications.

In certain embodiments, therapy management engine 114 determines a patient's SGLT2 inhibitor medication adherence and/or the patient's ideal SGLT2 inhibitor medication dose based on the patient's reabsorption threshold. Specifically, if a patient is prescribed a specific SGLT2 inhibitor medication dose, therapy management engine 114 determines the expected reabsorption threshold for the patient based on the patient's first reabsorption threshold and/or the reabsorption threshold of a population of patients taking a similar SGLT2 medication dose. If the patient is not taking the SGLT2 inhibitor medication appropriately, the SGLT2 inhibitor medication is dosed below an effective level for the patient, or the patient has become desensitized to the SGLT2 inhibitor medication, the patient may not experience the expected decrease in reabsorption threshold in response to the patient's SGLT2 inhibitor medication dose. In certain embodiments, therapy management engine 114 then provides feedback to the patient to take the SGLT2 medication as prescribed, increase SGLT2 inhibitor dose, or switch to an alternative medication which achieves the desired decrease in reabsorption threshold.

In certain embodiments, in addition to determining a presence of kidney disease and/or monitoring kidney function over time, therapy management engine 114 determines, based on the patient's reabsorption threshold, a diagnosis or risk of health complications related to kidney disease and/or diabetes. For example, therapy management engine 114 determines a risk or diagnosis of retinopathy and/or microvascular disease based on various additional analyte metrics, such as glucose time in range, or frequency of hyperglycemia. Based on the determined risk, therapy management engine 114 provides feedback to the patient to seek follow-up tests to confirm a risk of retinopathy and/or microvascular disease. In certain embodiments, if the patient is known to have a condition or disease that results in lower 1,5-AG levels and/or altered 1,5-AG levels such that 1,5-AG levels may not accurately portray kidney function, therapy management engine 114 determines the patient's kidney function cannot accurately be determined. For example, if, based on patient input, therapy management engine 114 determines the patient has terminal stage renal failure, is on dialysis, has advanced cirrhosis, is pregnant, is undergoing steroid therapy, etc., therapy management engine 114 determines the patient's kidney function cannot be accurately determined using 1,5-AG. In one example, the patient may consume 1-5AG supplements, to increase their 1-5AG levels in such examples to allow for accurate determination of kidney function even where the patient suffers from such conditions.

Method 401 of FIG. 4B is an example method for providing guidance to a patient to ensure that the patient's analyte levels are within range, either before or during method 400 or before or during method 500, for example in order to calculate the patient's reabsorption threshold.

Method 401 begins at block 402 by therapy management engine 114 monitoring one or more analyte levels of a patient with continuous analyte monitoring system 104. For example, continuous analyte monitoring system 104 comprises a continuous glucose and/or 1,5-AG sensor 202 to measure the patient's glucose and/or 1,5-AG levels. Further, therapy management engine 114 receives data from patient inputs. The patient inputs can be received in a variety of ways. For example, the inputs can be received or retrieved from patient profile 118, which includes demographic info 120, physiological info 122, disease progression info 124, medication info 126, inputs 130, metrics 132, etc. Inputs can also be received as patient input through the patient interface of a display device 107.

In certain embodiments, monitoring the patient's 1,5-AG levels and/or glucose levels can include optionally determining one or more 1,5-AG and/or glucose metrics, such as reabsorption threshold, rate of change, baseline, minimum or maximum, etc., based on the measured glucose and/or 1,5-AG levels. At block 406, therapy management engine 114 determines, based on glucose and/or 1,5-AG levels, that the patient's glucose levels are out of range (e.g., greater than 110 mg/dL, for example) and/or that the patient's 1,5-AG levels are out of range (e.g., below 25 micrograms/milliliter (μg/mL)) based on historical patient data and/or patient population data.

At block 410, therapy management engine 114 determines whether the patient's glucose levels are out of range (e.g., less than 70 mg/dL or greater than 110 mg/dL). If the patient's glucose levels are higher than the ideal range (e.g., based on the patient's historic glucose range and/or based on population data), therapy management engine 114 proceeds to block 420 to determine whether the patient has consumed food and/or a meal recently (e.g., within the last 3 hours, for example). If the patient has consumed food and/or a meal recently, therapy management engine 114 proceeds to block 422. At block 422, therapy management engine 114 returns to block 402 to continue monitoring glucose levels until glucose levels return to normal (e.g., less than 110 mg/dL).

Alternatively, if the patient has not consumed food and/or a meal recently, therapy management engine 114 proceeds to block 424. At block 424, therapy management engine 114 provides therapy management guidance to the patient to seek medical intervention for risk and/or presence of hyperglycemia/diabetes.

Returning to block 410, if the patient's glucose levels are lower than the ideal range (e.g., less than 70 mg/dL), therapy management engine 114 proceeds to block 426. At block 426, therapy management engine 114 recommends the patient consume complex carbohydrates, consume small amounts of glucose over time, and/or consume food or a meal followed by an insulin bolus to increase glucose levels. By consuming complex carbohydrates and/or following a meal with an optional insulin bolus (e.g., a slow acting insulin or a low dose of fast acting insulin), the patient can ensure glucose levels in the body increase gradually over time. By consuming small amounts of glucose over time, the amounts of glucose can be metered such that the patient can achieve specific glucose levels following each amount of glucose, such as 100 mg/dl, 110 mg/dL, 120 mg/dL, 130 mg/dL, 140 mg/dL, etc. In certain other embodiments, instead of a patient consuming glucose over time, glucose can be infused intravenously, for example, in order to control the patient's glucose levels as therapy management engine 114 determines a reabsorption threshold. In each of the above embodiments, a gradual increase in glucose allows therapy management engine 114 to accurately determine the patient's reabsorption threshold, once glucose and 1,5-AG levels are within range.

In certain other embodiments, therapy management engine 114 forecasts or extrapolates a patient's reabsorption threshold based on the rate of change of the patient's glucose and/or 1,5-AG levels. For example, if the patient's glucose levels are increasing too rapidly, therapy management engine 114 determines the patient's rate of change of glucose levels and the time at which 1,5-AG levels begin to decrease to determine the patient's reabsorption threshold (e.g., glucose level at which glucose begins to outcompete 1,5-AG for absorption). Additionally or alternatively, therapy management engine 114 forecasts or extrapolates a patient's reabsorption threshold based on the negative rate of change of glucose (e.g., glucose clearance rate) and 1,5-AG levels as glucose levels begin to return to baseline following an increase in glucose levels, such as when insulin begins to clear glucose from the body or when the patient completes an exercise session. For example, as glucose levels begin to decrease, in response to insulin or otherwise, the therapy management engine 114 determines the approximate glucose level at which 1,5-AG begins to be reabsorbed based on the time 1,5-AG levels begin to increase and the glucose clearance rate, therefore indicating the patient's reabsorption threshold.

In certain embodiments, therapy management engine 114 recommends the patient consume glucose to induce mild hyperglycemia in combination with insulin dosing, if necessary, to ensure glucose levels increase to a desired level gradually over time. Therapy management engine 114 continues monitoring glucose levels over time to determine when the patient's glucose levels are within the desired range.

Returning to block 410, if the patient's glucose levels are not out of range (e.g., within the desired range), therapy management engine 114 proceeds to block 428. At block 428, therapy management engine 114 determines whether the patient's 1,5-AG levels are low (e.g., below 25 μg/mL). If the patient's 1,5-AG levels are low, therapy management engine 114 proceeds to block 432. At block 432, therapy management engine 114 provides therapy management guidance to the patient to consume food and/or a supplement containing 1,5-AG. While 1,5-AG is not a critical compound to replenish in the body, 1,5-AG levels must be above a threshold (e.g., greater than 25 μg/mL, for example) in order to capture the patient's reabsorption threshold and, therefore, provide kidney disease therapy management guidance to the patient. Therapy management engine 114 continues monitoring the patient's 1,5-AG levels to determine when the patient's 1,5-AG levels return to the desired range.

From block 428, if the patient's 1,5-AG levels are not low (e.g., above 25 μg/mL), therapy management engine 114 returns to block 430. At block 430, therapy management engine 114 returns to block 406 to determine whether the patient's glucose levels and 1,5-AG levels are within range to monitor for the patient's reabsorption threshold.

In certain embodiments, the patient's 1,5-AG levels do not increase in response to consuming food and/or a supplement with 1,5-AG. Therapy management engine 114 then monitors the patient over time (e.g., over the course of 2 weeks) to determine whether the patient's 1,5-AG levels increase naturally. If the patient's 1,5-AG levels do not rise naturally over time, therapy management engine 114 determines the patient's kidney function is compromised, or that the patient is not consuming foods containing 1,5-AG. In certain embodiments, if the patient's 1,5-AG levels remain low (e.g., less than 25 μg/mL) over long periods of time (e.g., greater than 2 weeks), the patient excretes 1,5-AG more quickly than a healthy patient and/or the patient does not reabsorb 1,5-AG as quickly as a healthy patient, which indicates kidney dysfunction.

In certain embodiments, the patient's 1,5-AG variability over time can be higher than expected and/or increasing over time in patients with kidney disease. Further, a patient with compromised kidney function and/or kidney disease can have lower baseline 1,5-AG levels than a healthy patient. If therapy management engine 114 determines the patient's 1,5-AG levels demonstrate high variability over time and/or the patient's baseline 1,5-AG is lower than a healthy patient, therapy management engine 114 provides therapy management guidance to the patient that the patient's kidney function is compromised.

FIG. 5 describes an example method 500 for determining a filtration score for a patient and providing kidney disease therapy management guidance using an analyte monitoring system configured to measure at least 1,5-AG levels, according to certain embodiments of the present disclosure. Method 500 is described below with reference to FIGS. 1-3 and their components. As described in method 500, FIGS. 7A-9G and their components may be utilized to continuously monitor analyte data.

The method 500 begins at block 502, when therapy management engine 114 detects a decline in 1,5-AG levels. In certain embodiments, therapy management engine 114 detects a decline in 1,5-AG levels if the patient reaches a specified or defined reabsorption threshold, which indicates that the 1,5-AG levels of the patient are starting decline as 1,5-AG is cleared from the body instead of being reabsorbed through the kidney. In certain embodiments, as therapy management engine 114 continuously monitors 1,5-AG levels of the patient, a downward trend in 1,5-AG levels is detected or predicted. Therapy management engine 114 predicts or detects a downward trend in 1,5-AG levels based on historical information, based on the 1,5-AG levels indicating an onset of a negative rate of change, or based on 1,5-AG levels meeting a threshold of change from the baseline 1,5-AG level indicating a decrease in 1,5-AG.

Once therapy management engine 114 detects a decline in 1,5-AG levels at block 502, therapy management engine 114 continues to block 504. At block 504, therapy management engine 114 initiates a monitoring period at time T₀. In certain embodiments, therapy management engine 114 can instruct the patient to fast overnight prior to initiating the monitoring period to ensure the patient's glucose level reaches a baseline level and to ensure the patient's 1,5-AG level is at a stable baseline level (e.g., a maximum). Therapy management engine 114 then instructs the patient to ingest a specified amount of glucose to increase the patient's glucose level to a known level (e.g., at or above the patient reabsorption threshold). The known level can be the glucose level at which glucose begins to outcompete 1,5-AG, thereby causing 1,5-AG to begin to clear and 1,5-AG levels to decrease. In certain embodiments, the filtration score calculation is done via a urine sample. In such an example, at T₀, the patient is prompted to provide a urine sample. This urine sample may be discarded, as it serves the function of creating the baseline for the patient in the body, for the future urine sample taken in later steps.

At block 506, therapy management engine 114 determines a first level of 1,5-AG (e.g., the starting level of 1,5-AG) at T₀. For example, if the analyte measurements are periodic, the first level of 1,5-AG would be the 1,5-AG measurement at the time closest to T₀, such as the 1,5-AG measurement at T₀, right before T₀, or after T₀. In certain embodiments, an average of one or more 1,5-AG periodic measurements for a certain number of measurements (e.g., 3 to 5 measurements) over a time period that includes the time T₀ are used to calculate the first level of 1,5-AG that corresponds to the time T₀.

The method 500 then continues to block 508, where therapy management engine 114 monitors the 1,5-AG levels of the patient on a periodic basis (e.g., every 1 minute, 5 minutes, 10 minutes, etc.). In addition, in certain embodiments, therapy management engine 114 determines a rate of change of 1,5-AG on a periodic basis, which can be the same as or different from the periodic basis of the 1,5-AG level monitoring.

At block 510, therapy management engine 114 determines a second level of 1,5-AG at a second point in time, T₁, based on determining that a predetermined amount of 1,5-AG has cleared from the body. For example, T₁ can be at or before a point in time where the patient's 1,5-AG level meets a threshold level indicating that 1,5-AG has been cleared from the body (e.g., 0 μg/mL, lower than 10 μg/mL, or another predefined threshold). In certain embodiments, T₁ is a time at which a measured rate of change of decline of 1,5-AG is lower than a threshold rate of change (e.g., a rate of change of 0 or near 0, or less than 5 μg/mL per minute) indicating a slowing of the rate of change of decline. The slowing of the rate of change of decline indicates 1,5-AG clearance has slowed or stopped.

Alternatively or additionally, T₁ can be based on glucose levels measured during the monitoring period. As described above, when glucose levels reach a certain level (e.g., the reabsorption threshold), glucose outcompetes 1,5-AG for reabsorption by the kidney. Thus, at this certain level of glucose, 1,5-AG is cleared from the body and a decrease in the level of 1,5-AG is observed. Accordingly, once the decrease in 1,5-AG levels has been detected, therapy management engine 114 can begin to monitor glucose levels. If therapy management engine 114 detects a decrease in glucose levels toward the reabsorption threshold for the patient, then T₁ is a time prior to when the glucose levels begin to decrease in order to capture 1,5-AG levels at a time prior to when 1,5-AG begins to be reabsorbed by the kidney into the bloodstream (e.g., 1,5-AG levels would begin stabilize).

In another example, T₁ is fixed at a predefined time after T₀. For example, T₁ can be fixed at 15 minutes, 20 minutes, 30 minutes, or any similar time period from T₀. In another example, T₁ is any time after T₀ where therapy management engine 114 detects a certain decrease in 1,5-AG levels from T₀. For example, in the case where a urine test is being performed, T₁ is the time when a urine sample is taken. For example, therapy management engine 114 prompts the patient to provide a urine sample at T₁, where T₁ is calculated based on any of the methods mentioned herein. In certain embodiments, therapy management engine 114 instructs the patient to maintain their glucose level or to increase their glucose level from the glucose level at T₀, for the entire period between T₀ and T₁ to ensure that the 1,5-AG is clearing from the body rather than being reabsorbed for the entire period between T₀ and T₁. For example, therapy management engine 114 can instruct the patient to consume a glucose drink or a glucose supplement to maintain the patient's glucose level or increase the patient's glucose level. Alternatively, therapy management engine 114 can instruct the patient to take a medication (e.g., SGLT2 inhibitor) to cause 1,5-AG to clear from the body regardless of the glucose levels of the patient.

Once therapy management engine 114 determines T₁, therapy management engine 114 determines the second level of 1,5-AG at T₁. For example, since the 1,5-AG measurements are periodic, the 1,5-AG level at the time closest to T₁, such as the 1,5-AG measurement at T₁, right before T₁, or after T₁. In certain embodiments, an average of one or more 1,5-AG periodic measurements for a certain number of measurements (e.g., 3 to 5 measurements) over a time period that includes T₁ are used to calculated the second level of 1,5-AG that corresponds to T₁.

At block 512, therapy management engine 114 calculates a value representing the total cleared 1,5-AG based on the difference between the first level of 1,5-AG corresponding to T₀ and the second level of 1,5-AG corresponding to T₁ calculated in blocks 506 and 510 respectively. Alternatively, or additionally, where a urine sample was collected at T₁, the urine test is used to measure a total cleared amount of 1,5-AG.

Next, at block 514, therapy management engine 114 calculates a total mass of 1,5-AG cleared from the body through the urine. The volume of 1,5-AG is calculated either (1) based on the volume of the urine where a urine test is utilized or (2) based on the total body water volume of the patient where a urine test is not utilized.

Where a urine test is utilized, the total mass of 1,5-AG is the urine concentration of 1,5-AG at T₁ multiplied by the volume of urine collected.

Where a urine test is not utilized, the total mass of 1,5-AG is based on the first level of 1,5-AG at T₀ minus the second level of 1,5-AG at T₁ multiplied by the total body water volume.

The total body water volume is an estimation of the volume of water available in the body and can be calculated using a body mass of the patient, an age factor multiplier and a gender factor multiplier. Other multipliers can also be included to account for other factors that can account for differences in the ratio of water to body weight in the patient. Equation 3 is an example total body water volume estimation (TBW) based on the patient's weight alone, Equation 4 is an example TBW calculation in men, and Equation 5 is an example TBW calculation in women:

$$T B W = weight * 0.58 \qquad Eq . 3$$

$$T B W = 2.447 - ( 0.09156 * Age ) + ( 0.1074 * Height ) + ( 0.3362 * Weight ) \qquad Eq . 4$$

$$T B W = ( 0.1069 * Height ) + ( 0.2466 * Weight ) - 2.097 \qquad Eq . 5$$

At block 516, therapy management 114 determines the filtration score of the patient. The filtration score of the patient is determined by (1) dividing the total volume of 1,5-AG cleared, as determined at block 514, by the average 1,5-AG level from T₀ and T₁ and then (2) dividing the output of (1) by the time between T₀ and T₁. The filtration score can be representative of or similar to a GFR score currently measured at a lab. The filtration score calculated using real-time analyte levels can be more effective by using analyte levels that are averaged over time, or selected at optimal times, based on observed patterns for the patient.

At block 518, therapy management engine 114 provides therapy management guidance to the patient based on the filtration score of the patient. For example, therapy management engine 114 can display the filtration score to the patient at display device 107, can use the filtration score to plot a graph to be provided to the patient at display device 107, and can use the filtration score to provide therapy management guidance to the patient. The filtration score is an indicator of kidney health for the patient and, therefore, a calculation of the filtration score multiple times over a specified period of time (e.g., one week, one month, or another time period) can be used to calculate an average filtration score that is displayed to the patient as the filtration score for the patient or used to provide therapy management guidance to the patient.

The filtration score can further be plotted and/or stored in a memory to be compared to one or more filtration scores over various time periods to detect a pattern. The detected pattern can be indicative of worsening or improved kidney function. For example, if the patient shows a higher filtration score over time, therapy management engine 114 determines the patient's kidney function is improving. Alternatively, if the patient shows a slower filtration rate over time, therapy management engine 114 determines the patient's kidney function has worsened. This determination of the patient's kidney function over time can be displayed to the patient via display device 107.

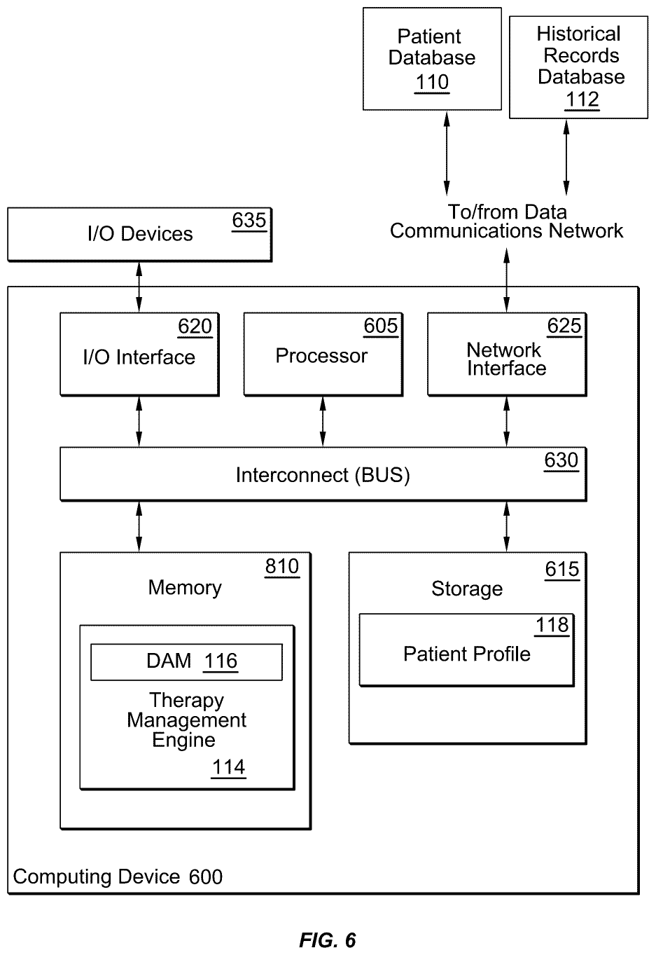

FIG. 6 is a block diagram depicting a computing device 600 configured to execute a therapy management engine (e.g., therapy management engine 114), according to certain embodiments disclosed herein. Although depicted as a single physical device, in embodiments, computing device 600 can be implemented using virtual device(s), and/or across a number of devices, such as in a cloud environment. As illustrated, computing device 600 includes a processor 605, memory 610, storage 615, a network interface 625, and one or more I/O interfaces 620. In the illustrated embodiment, processor 605 retrieves and executes programming instructions stored in memory 1010, as well as stores and retrieves application data residing in storage 615. Processor 605 is generally representative of a single CPU and/or GPU, multiple CPUs and/or GPUs, a single MCU, CPU and/or GPU having multiple processing cores, and the like. Memory 610 is generally included to be representative of a random-access memory. Storage 615 can be any combination of disk drives, flash-based storage devices, and the like, and can include fixed and/or removable storage devices, such as fixed disk drives, removable memory cards, caches, optical storage, network attached storage (NAS), or storage area networks (SAN).

In some embodiments, input and output (I/O) devices 635 (such as keyboards, monitors, etc.) can be connected via the I/O interface(s) 620. Further, via network interface 625, computing device 600 can be communicatively coupled with one or more other devices and components, such as patient database 110. In certain embodiments, computing device 600 is communicatively coupled with other devices via a network, which can include the Internet, local network(s), and the like. The network can include wired connections, wireless connections, or a combination of wired and wireless connections. As illustrated, processor 605, memory 610, storage 615, network interface(s) 625, and I/O interface(s) 620 are communicatively coupled by one or more interconnects 630. In certain embodiments, computing device 600 is representative of display device 107 associated with the patient. In certain embodiments, as discussed above, the display device 107 can include the patient's laptop, computer, smartphone, and the like. In another embodiment, computing device 600 is a server executing in a cloud environment.

In the illustrated embodiment, storage 615 includes patient profile 118. Memory 610 includes therapy management engine 114, which itself includes DAM 116. Therapy management engine 114 is executed by computing device 600 to perform operations in method 300 of FIG. 3B, method 400 of FIG. 4A, method 401 of FIG. 4B, and/or operations of method 500 in FIG. 5.

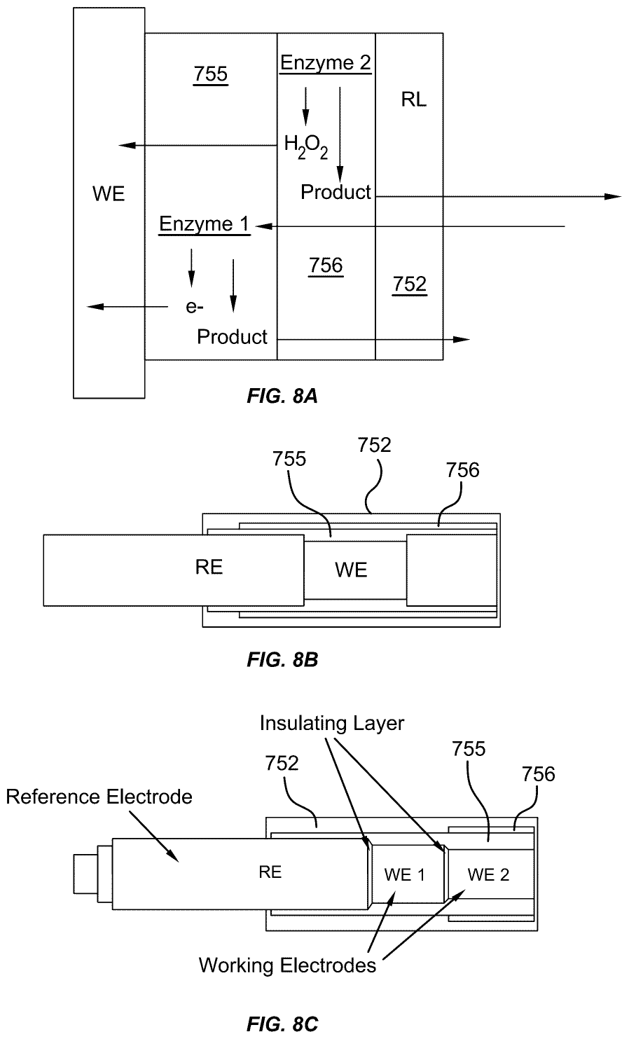

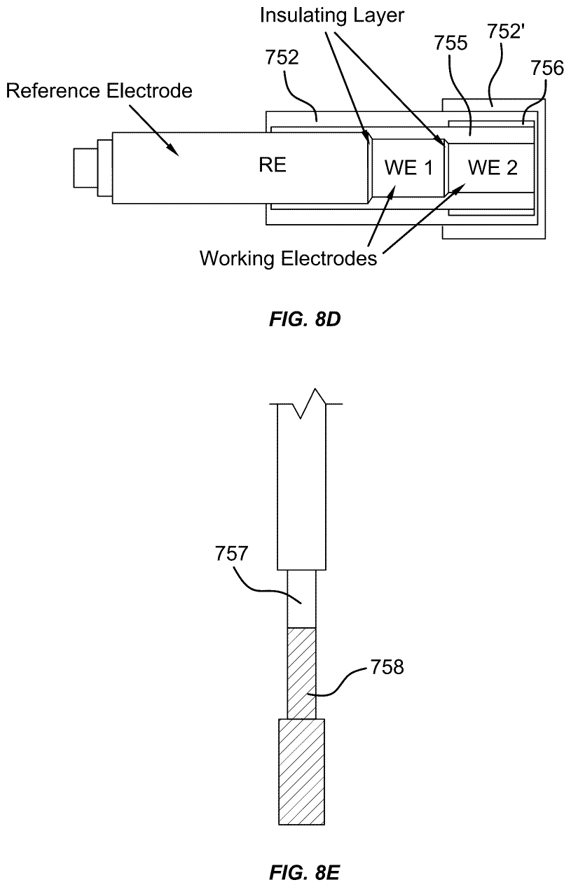

As described above, continuous analyte monitoring system 104, described in relation to FIG. 1 can be a multi-analyte sensor system including a multi-analyte sensor. FIGS. 7-8 describe example multi-analyte sensors used to measure multiple analytes.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, an ionophore, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, pyranose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection can be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoA mps of current for every 1 mg/dl of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include adjustment or measurement of current and/or voltage, inductance, capacitance, impedance, transconductance, and charge. Optical properties include absorbance, fluorescence/phosphorescence, fluorescence/phosphorescence decay rate, wavelength shift, dual wave phase modulation, bio/chemiluminescence, reflectance, light scattering, Raman shift, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more single or double stranded DNA or RNA moieties. Transcutaneous continuous multi-analyte sensors can be used in vivo over various lengths of time. The continuous multi-analyte sensor systems discussed herein can be transcutaneous devices, in that a portion of the device can be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, electrodeposition, plasma polymerization, electrospraying), or thick film techniques (spray coating, dip coating, spin coating, pad printing, screen printing, discrete dispense, inkjet deposition, slot-die coating, brush coating, film coating, drop-let coating, and the like). Additional steps can be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (μm), or less, to about 1, 2, 3, 4, 5, 6, 7, 8, 9, 10, 11, 12, 13, 14, 15, or 16 μm is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Y et in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl) carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Y et in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro) chemical reactions involved in sensing, but forms chemical bonds with the (electro) chemically active components of the membrane. In some examples these bonds are covalent. In one example, a tether can be formed in solution prior to one or more interlayers of a membrane being formed, where the tether bonds two (electro) chemically active components directly to one another or alternately, the tether(s) bond (electro) chemically active component(s) to polymeric backbone structures. In another example, (electro) chemically active components are comixed along with crosslinker(s) with tunable lengths (and optionally polymers) and the tethering reaction occurs as in situ crosslinking. Tethering can be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as electrodeposition, plasma polymerization, electrospraying, spray coating, dip coating, casting, electrospinning, vapor deposition, spin coating, pad printing, screen printing, discrete dispense, inkjet deposition, solt-die coating, brush coating, film coating, droplet coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure or otherwise cross-link by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties, including the hydrophilic and hydrophobic domains dispersed throughout the film. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly (ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spray coating, self-assembling monolayers (SAMs), painting, dip coating, vapor depositing, electrodepositing, plasma polymerizing, electrospraying, pad printing, spin coating, discrete dispensing, inkjet depositing, slot-die coating, molding, 3-D printing, lithographic techniques (e.g., photolithograph), micro- and nano-pipetting printing techniques, screen printing, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor can promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein can include a biointerface layer. The biointerface layer, like the drug releasing layer, can include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein can include a drug releasing membrane at least partially functioning as or in combination with a biointerface membrane. The drug releasing membrane can include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(I-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Embodiments of the present disclosure advantageously provide continuous multi-analyte sensors with various membrane configurations suitable for facilitating signal transduction corresponding to analyte concentrations, either simultaneously, intermittently, and/or sequentially. In one example, such sensors can be configured using a signal transducer, comprising one or more transducing elements. Such continuous multi-analyte sensors can employ various transducing means and methods, such as amperometric, voltametric, chronoamperometric, coulometric, chronocoulometric, potentiometric, conductance, and impedimetric methods, among other techniques.

In one example, the transducing element comprises one or more membranes that can comprise one or more layers and or domains, each of the one or more layers or domains can independently comprise one or more signal transducers, e.g., enzymes, ionophores, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain can comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte/oxidase enzyme pairs: for example, glucose/glucose oxidase, pyranose/pyranose oxidase, alcohol/alcohol oxidase, cholesterol/cholesterol oxidase, glactose: galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine: xanthine/xanthine oxidase, lactate/lactate oxidase, L-amino acid oxidase, catechol/catechol oxidase, beta-hydroxybutyrate/beta-hydroxybutyrate oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, enzyme cascades, dimerized and/or fusion enzymes.

### NAD Based Multi-Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)⁺/and NAD(P)H forms essentially without being consumed. NAD(P)⁺ and NAD(P)H are electroactive and can undergo reduction and oxidation reactions, respectively, at a suitable bias potential.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, the redox-active cofactors NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NA D+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, glucose (glucose dehydrogenase), pyranose (pyranose dehydrogenase), ketones (beta-hydroxybutyrate dehydrogenase), glycerol (glycerol dehydrogenase), cortisol (11β-hydroxysteroid dehydrogenase), alcohol (alcohol dehydrogenase), aldehydes (aldehyde dehydrogenase), pyranose (pyranose oxidase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 7A. With reference to FIG. 7B, one or more optional layers can be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 7A-7B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, methylene blue, or nitrosoanilines are used. In another example, metallo-organic mediators, such as ruthenium-phenanthroline-dione or osmium (bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

Another example of a continuous ketone analyte detection configuration employing electrode-associated mediator-coupled diaphorase/NAD+/dehydrogenase is depicted below:

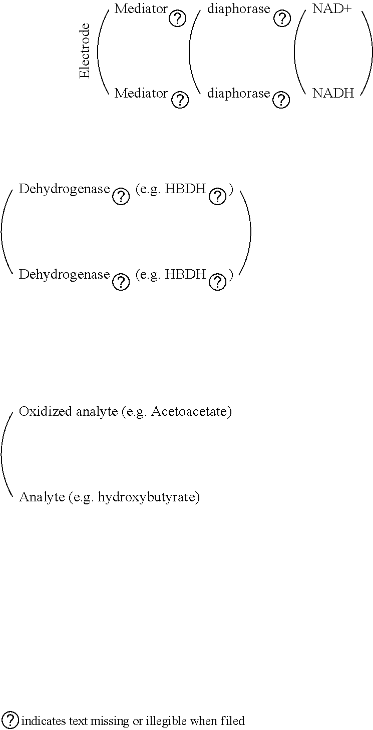

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ can be covalently coupled to an aspect of the enzyme domain having a higher molecular weight than the NAD+ which can improve a stability profile of the NAD+, improving the ability to retain and/or immobilize the NAD+ in the enzyme domain. For example, dextran-NAD.

In one example, the sensing region comprises one or more NADH: acceptor oxidoreductases and one or more NA D-dependent dehydrogenases. In one example, sensing region comprises one or more NADH: acceptor oxidoreductases and one or more NAD(P)-dependent dehydrogenases with NAD(P)+ or NAD(P)H as cofactors present in sensing region. In one example, the sensing region comprises an amount of diaphorase.

In one example, a single analyte sensor configuration suitable for combination with another analyte sensor configuration is provided. Thus, an EZL layer of about 1-20 μm thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400M W], about 20 uL 500 mg/ml diaphorase, about 40 uL 250 mg/ml poly vinyl imidazole-osmium bis(2,2′-bipyridine) chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os (bpy)2Cl and about 1-12% PEG-DGE (400 MW). The substrates discussed herein that can include working electrodes can be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous analyte sensor as depicted in FIGS. 7A-7B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) can also contain a polymer or protein binder, such as zwitterionic polyurethane, and/or albumin. Alternatively, in addition to NAD(P)H, the membrane can contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and can be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NA DH oxidase. In one example, NA DH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous analyte sensor configurations can be adapted to other analytes or used in combination with other sensor configurations. For example, analyte(s)-dehydrogenase enzyme combinations can be used in any of the membranes of the sensing region include; glucose (glucose dehydrogenase); pyranose (pyranose dehydrogenase); glycerol (glycerol dehydrogenase); cortisol (11β-hydroxysteroid dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semi permeable membrane can be an ion selective membrane selective for an ion analyte of interest, such as ammonium ion.

In another example, a continuous multi-analyte sensor configuration comprising one or more enzymes and/or at least one cofactor was prepared. FIG. 7C depicts this exemplary configuration, of an enzyme domain 750 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 751 comprising an amount of cofactor is positioned adjacent the first enzyme domain. The amount of cofactor in the second membrane can provide an excess for the enzyme, e.g., to extend sensor life. One or more resistance domains 752 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 7D depicts an alternative enzyme domain configuration comprising a first membrane 751 with an amount of cofactor that is positioned more proximal to at least a portion of a WE surface. Enzyme domain 750 comprising an amount of enzyme is positioned adjacent the first membrane.

In the membrane configurations depicted in FIGS. 7C-7D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as M g⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

### Glucose Sensor Configurations

In certain embodiments, continuous glucose sensor device configurations are provided. In one example, glucose oxidase (GOx) can be included in one or more enzyme domains and positioned adjacent the working electrode surface. The catalysis of the glucose using GOx, in the presence of dissolved oxygen, produces hydrogen peroxide which can be detected and/or measured qualitatively or quantitatively, using amperometric, voltametric, chronoamperometric, coulometric, chronocoulometric, potentiometric, conductance, and impedimetric methods.

In certain embodiments, the sensing region for the enzyme substrate-oxidase enzyme configurations has one or more enzyme domains which include one or more electrodes. In certain embodiments, the sensing region has one or more enzyme domains, with or without one or more electrodes, and one or more interference blocking membranes (e.g., permselective membranes, and/or charge exclusion membranes). The one or more interference blocking membranes can attenuate one or more interferents from diffusing through the membrane to the working electrode. In certain embodiments, the sensing region has one or more enzyme domains, with or without the one or more electrodes, and one or more resistance domains, with or without the one or more interference blocking membranes. In certain embodiments, the sensing region has one or more enzyme domains, with or without the one or more electrodes, one or more resistance domains, with or without the one or more interference blocking membranes, and one or more biointerface membranes and/or drug releasing membranes. The one or more biointerface membranes and/or drug releasing membranes attenuate the diffusion of one or more analytes or enzyme substrates and attenuate the immune response of the host after insertion.

In one example, the one or more interference blocking membranes are deposited on a surface anterior to the working electrode and/or the electrode surface. In one example, the one or more interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing through the sensing region. Such membranes can be used to attenuate glucose itself as well as attenuate other electrochemically actives species or other analytes that can otherwise interfere by producing a signal if they diffuse to the working electrode. Said membranes can be engineered to allow glucose permeation to occur relatively unabated. Said permselectivity properties can be based on molecular weight.

In one example, the working electrode used comprised platinum and the potential applied is about 0.6 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of glucose. When appropriately designed to obey stoichiometric behavior, the presence of a specific concentration of glucose should cause a commensurate reduction in local oxygen in a direct (linear) relation with the concentration of glucose. Accordingly, a multi-analyte sensor for both glucose and oxygen can therefore be provided.

In another example, the above mentioned glucose sensor configuration can include one or more mediators. In one example, the one or more mediators are present in, on, or about one or more electrodes or electrode surfaces and/or are deposited or otherwise associated with the surface of the WE or reference electrode (“RE”). In one example, the one or more mediators eliminate or reduce direct oxidation of interfering species that can reach the WE or RE. In one example, the one or more mediators provide a lowering of the operating potential of the WE/RE, for example, from about 0.6V to about 0.3V or less on a platinum electrode, which can reduce or eliminate oxidation of endogenous interfering species. Examples of one or mediators are provided above, such as methylene blue, polyvinylimidizole-Os(bpy)₂Cl, etc. Other electrodes, such as counter electrodes, can be employed.

In one example, other enzymes or additional components can be added to the polymer mixture(s) that constitute any part of the sensing region to increase the stability of the aforementioned sensor and/or reduce or eliminate the byproducts of the glucose/glucose oxidase reaction. In another example, the additional components can also be proteins (e.g., bovine serum albumin (BSA)) or cross-linkers (e.g., glutaraldehyde, N,N′-Carbonyldiimidazole (CDI)). Increasing stability includes storage or shelf life and/or operational stability (such as retention of enzyme activity during use). For example, byproducts of enzyme reactions can be undesirable for increased shelf life and/or operational stability, and can thus be desirable to reduce or remove. In one example, xanthine oxidase can be used to remove byproducts of one or more enzyme reactions.

In another example, a dehydrogenase enzyme is used with an oxidase for the detection of glucose alone or in combination with oxygen. Thus, in one example, glucose dehydrogenase is used to oxidize glucose to Glucono-δ-lactone in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled glucose dehydrogenase (GDH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ can be coupled to a polymer, such as dextran, and the polymer immobilized in the enzyme domain along with GDH. This provides for retention of the co-factor and availability thereof for the active site of GDH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface can be provided. A chemical moiety capable of assisting with electron transfer from the enzyme or cofactor to the electrode surface can be used and includes one or more mediators as described below.

In one example, any one of the aforementioned continuous glucose sensor configurations are combined with any one of the aforementioned continuous analyte monitoring configurations to provide a continuous multi-analyte sensor device as further described below. In one example, a continuous glucose monitoring configuration combined with any one or more of the aforementioned continuous analyte sensor configurations to provide a continuous multi-analyte sensor device as further described below.

### Pyranose Sensor Configurations

In another example, a continuous pyranose sensor device configuration is provided. Thus, in one example, pyranose oxidase (POx) can be included in one or more enzyme domains and positioned adjacent the working electrode surface. In the presence of oxygen, the catalysis of the pyranose using POx, produces hydrogen peroxide which can be detected and/or measured qualitatively or quantitatively, using, among other techniques, amperometry, voltametric, chronoamperometric, coulometric, chronocoulometric, potentiometric, conductance, and impedimetric methods.

In certain embodiments, the sensing region for the enzyme substrate-oxidase enzyme configurations has one or more enzyme domains which include one or more electrodes. In certain embodiments, the sensing region has one or more enzyme domains, with or without one or more electrodes, and one or more interference blocking membranes (e.g., permselective membranes, and/or charge exclusion membranes). The one or more interference blocking membranes can attenuate one or more interferents from diffusing through the membrane to the working electrode. In certain embodiments, the sensing region has one or more enzyme domains, with or without the one or more electrodes, and one or more resistance domains, with or without the one or more interference blocking membranes. In certain embodiments, the sensing region has one or more enzyme domains, with or without the one or more electrodes, one or more resistance domains, with or without the one or more interference blocking membranes, and one or more biointerface membranes and/or drug releasing membranes. The one or more biointerface membranes and/or drug releasing membranes attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate pyranose itself as well as attenuate other electrochemically actives species or other analytes that can otherwise interfere by producing a signal if they diffuse to the working electrode.

In one example, the working electrode used comprised platinum and the potential applied is about 0.6 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of pyranose. When appropriately designed to obey stoichiometric behavior, the presence of a specific concentration of pyranose should cause a commensurate reduction in local oxygen in a direct (linear) relation with the concentration of pyranose. Accordingly, a multi-analyte sensor for both pyranose and oxygen can therefore be provided.

In another example, the above-mentioned pyranose sensor configuration can include one or more mediators. In one example, the one or more mediators are present in, on, or about one or more electrodes or electrode surfaces and/or are deposited or otherwise associated with the surface of the WE or RE. In one example, the one or more mediators eliminate or reduce direct oxidation of interfering species that can reach the WE or RE. In one example, the one or more mediators provide a lowering of the operating potential of the WE/RE, for example, from about 0.6V to about 0.3V or less on a platinum, gold, or carbon electrode, which can reduce or eliminates oxidation of endogenous interfering species. Examples of suitable mediators are provided below. Other electrodes, e.g., counter electrodes, can be employed.

In one example, other enzymes or additional components can be added to the polymer mixture(s) that constitute any part of the sensing region to increase the stability of the aforementioned sensor and/or reduce or eliminate the byproducts of the pyranose/pyranose oxidase reaction. In certain embodiments, the enzymes or additional components that can be added to increase stability can be cross-linked polymers and/or enzyme stabilizers. Increasing stability includes storage or shelf life and/or operational stability (e.g., retention of enzyme activity during use). For example, byproducts of enzyme reactions can be undesirable for increased shelf life and/or operational stability, and can thus be desirable to reduce or remove. In one example, xanthine oxidase can be used to remove byproducts of one or more enzyme reactions.

In another example, a dehydrogenase enzyme is used with an oxidase for the detection of pyranose alone or in combination with oxygen. Thus, in one example, pyranose dehydrogenase is used to oxidize pyranose to 2-dehydro-pyranose in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor. Said NAD(P)H, in certain configurations, can be oxidized directly with the application of a suitable bias potential.

In the aforementioned dual enzyme configuration, a signal can be sensed either by: (1) an electrically coupled pyranose dehydrogenase (PDH), for example, using an electro-active hydrogel polymer comprising one or more mediators; (2) oxygen electrochemical sensing to measure the oxygen consumption of the NA DH oxidase; or (3) hydrogen peroxide electrochemical sensing to measure the hydrogen peroxide generation by means of NADH oxidase in the presence of NADH and oxygen. In an alternative example, the co-factor NAD(P)H or NAD(P)+ can be coupled to a polymer, such as dextran, the polymer immobilized in the enzyme domain along with PDH. This provides for retention of the co-factor and availability thereof for the active site of PDH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface can be provided. A chemical moiety capable of assisting with electron transfer from the enzyme or cofactor to the electrode surface can be used and includes one or more mediators as described below.

In one example, any one of the aforementioned continuous pyranose sensor configurations are combined with any one of the aforementioned continuous analyte monitoring configurations to provide a continuous multi-analyte sensor device as further described below. In one example a continuous pyranose monitoring configuration combined with the aforementioned continuous glucose sensor configuration to provide a continuous multi-analyte sensor device as further described below.

### One-Working-Electrode Configurations for Dual Analyte Detection

In one example, at least a dual enzyme domain configuration in which each layer contains one or more specific enzymes and optionally one or more cofactors is provided. In a broad sense, one example of a continuous multi-analyte sensor configuration is depicted in FIG. 8A where a first membrane 755 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 756 (EZL2) with at least one second enzyme (Enzyme 2) is positioned adjacent 755 ELZ1, and is generally more distal from WE than EZL1. One or more resistance domains (RL) 752 can be provided adjacent EZL2 756, and/or between EZL1 755 and EZL2 756. The different enzymes catalyze the transformation of the same analyte, but at least one enzyme in EZL2 756 provides hydrogen peroxide and the other at least one enzyme in EZL1 755 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 8A, a first analyte diffuses through RL 752 and into EZL2 756 resulting in generation of hydrogen peroxide via interaction with Enzyme 2. Hydrogen peroxide diffuses at least through EZL1 755 to WE and transduces a signal that corresponds directly or indirectly to the first analyte concentration. A second analyte, which is different from the first analyte, diffuses through RL 752 and EZL2 756 and interacts with Enzyme 1, which results in electron transfer to WE and transduces a signal that corresponds directly or indirectly to the second analyte concentration.

As shown in FIG. 88, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 756 providing hydrogen peroxide and the at least other enzyme in EZL1 755 not providing hydrogen peroxide, e.g., providing electron transfer to the WE surface corresponding directly or indirectly to a concentration of the analyte.

In one example, an inner layer of the at least two enzyme domains EZL1, EZL2 755, 756 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 755 is more proximal to the WE surface and can have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 755 is directly adjacent the WE.

The second layer of at least dual enzyme domain (the outer layer EZL2 756) of FIG. 8B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 756 and through the inner layer EZL1 755 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry, pulsed amperometric detection, linear sweep voltammetry, differential pulse voltammetry, square wave voltammetry, etc. In some examples, impedimetric sensing can be used. In one example, a phase shift (e.g., a time lag) can result from detecting two signals from two different working electrodes, each signal being generated by a different EZL (EZL1, EZL2, 755, 756) associated with each electrode. The two (or more) signals can be broken down into components to detect the individual signal and signal artifacts generated by each of EZL1 755 and EZL2 756 in response to the detection of two analytes. In some examples, each EZL detects a different analyte. In other examples, both EZLs detect the same analyte.

In another alternative exemplary configuration, as shown in FIGS. 8C-8D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is pyranose.

Thus, FIGS. 8C-8D depict exemplary configurations of a continuous multi-analyte sensor construct in which EZL1 755, EZL2 756 and RL 752 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 8C-8D, WE1 represents a first working electrode surface configured to operate at P1, WE2 represents a second working electrode surface configured to operate at P2, WE1 is electrically insulated from WE2, and RE represents a reference electrode electrically isolated from both WE1, WE2. One resistance domain is provided in the configuration of FIG. 8C that covers the RE and WE1, WE2. An additional resistance domain is provided in the configuration of FIG. 8D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (hydrogen peroxide producing producing) and glucose dehydrogenase (electrically coupled) configuration. Measurement of glucose at two potentials and from two different electrodes provides more data points and accuracy. Such approaches can not be needed for glucose sensing, but can be applied across the biomarker sensing spectrum of other analytes, alone or in combination with glucoses sensing, such as pyranose sensing and glucose/pyranose sensing.

In an alternative configuration of that depicted in FIGS. 8C-8D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 8D, such an arrangement of RL's is depicted, where an additional RL 752′ is adjacent WES2 but substantially absent from WES1.

In one example of measuring two different analytes, the above configuration comprising enzyme domain EZL1 755 comprising one or more enzyme(s) and one or more mediators for at least one enzyme of EZL1 to provide for direct electron transfer to the WES1 and determining a concentration of at least a first analyte. In addition, enzyme domain EZL2 756 can comprise at least one enzyme that provides peroxide (e.g., hydrogen peroxide) or consumes oxygen during catalysis with its substrate. The peroxide or the oxygen produced in EZL2 756 migrates to WES2 and provides a detectable signal that corresponds directly or indirectly to a second analyte. For example, ELZ1 755 can be pyranose oxidase, ELZ2 756 can be glucose oxidase, and WES2 can be carbon, wired to glucose oxidase to measure glucose, while WES1 can be platinum, that measures peroxide produced from pyranose oxidase/pyranose in EZL1 755. The combinations of electrode material and enzyme(s) as disclosed herein are examples and non-limiting.

In one example, the potentials of P1 and P2 can be separated by an amount of potential so that both signals (from direct electron transfer from EZL1 755 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., pulsed amperometric detection. In one example, an alternative configuration is provided but hydrogen peroxide production in EZL2 is replaced by another suitable electrolysis compound that maintains the P2+P1 relationship, such as oxygen, and at least one enzyme-substrate combination that provide the other electrolysis compound.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 8E, an example of such composite electrode surfaces is shown, in which an extended platinum covered wire 757 is half coated with carbon 758, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that can be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include pyranose (e.g., 1,5-AG) sensing (pyranose dehydrogenase electrically coupled enzyme in EZL1 755) and glucose sensing (glucose oxidase in EZL2 756). Other membranes can be used in the aforementioned configuration, such as electrode, resistance, bio-interfacing, and drug releasing membranes. In other examples, one or both of the working electrodes (WE1, WE2) can be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein can instead or additionally include graphene, graphene oxide, or other materials suitable for forming the working electrodes, such as commercially available carbon ink.

### Planar Analyte Sensors

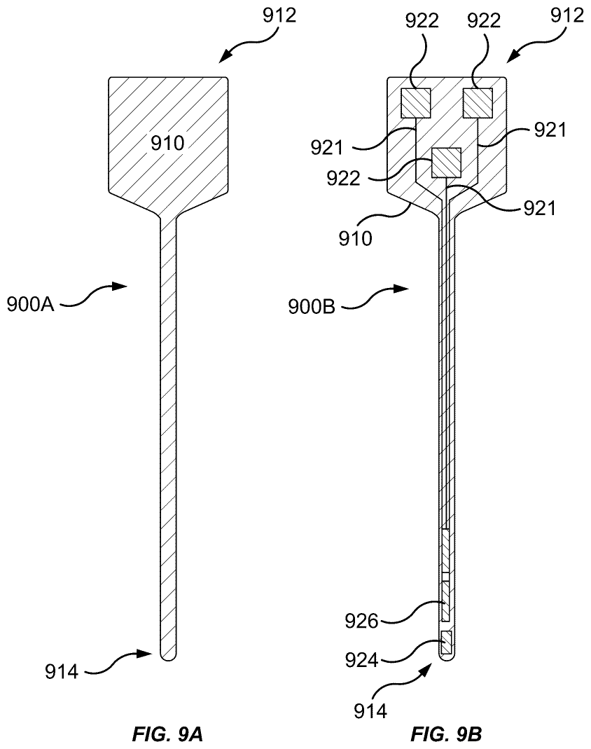

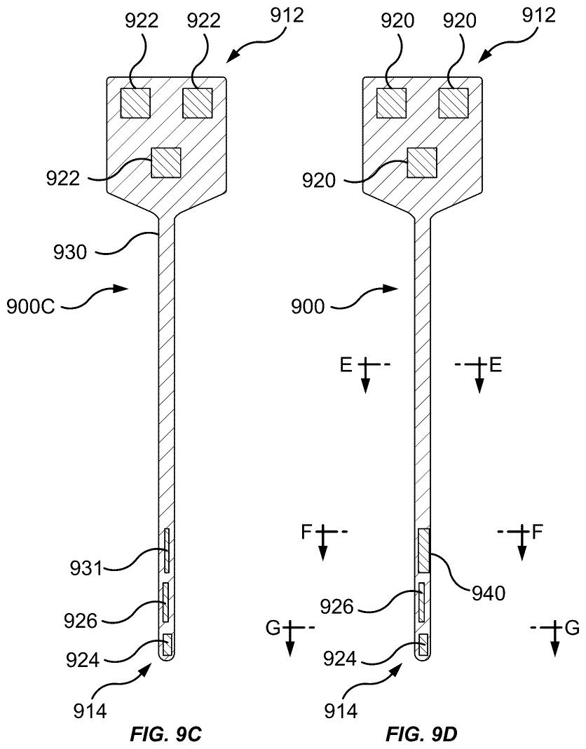

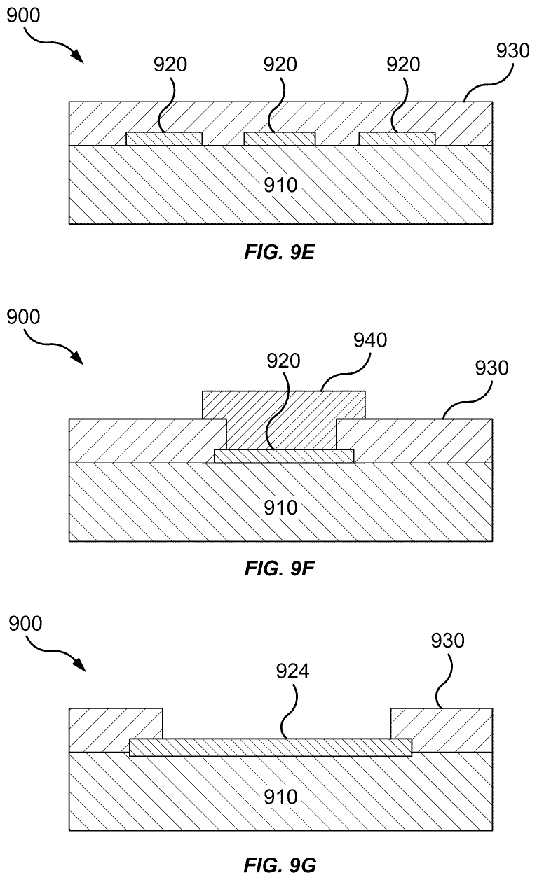

FIGS. 9A-9G depict schematic diagrams of planar analyte sensors. Each of the planar analyte sensors discussed herein can be configured to measure concentrations of one or more analytes. Planar analyte sensors can be readily manufactured and create reproducible results. Planar analyte sensors can be configured to monitor, including to continuously monitor, at least one analyte, and, in some examples, two or more analytes. The planar analyte sensors can be configured differently and can be described based on the geometry of their electrode layouts. The sensor types can include single-sided or double-sided layouts. In single-sided layouts as depicted in FIGS. 9A-9G, the electrodes can be conductive traces and can be in a co-planar arrangement, a stacked arrangement, or a staggered arrangement. In double-sided layouts, the electrodes can be in a co-planar arrangement (aligned along a shared plane in a single layer along each substrate side), a stacked arrangement (aligned along a shared plane perpendicular to the substrate side(s)), or a staggered arrangement (offset along or more plane or axis), as well as arrangements where connector pads are on a single side of the sensor, or arrangements where connector pads are on both sides of the sensor.

FIGS. 9A-9G illustrate a single sided co-planar analyte sensor assembly 900, in accordance with an example. The sensor assembly can have a first end 912 and a second end 914. The sensor assembly 900 can include substrate 910, conductive traces 921, connector pads 922, working electrode 924, counter electrode 926, insulator 930, and reference electrode 940. In sensor assembly 900, a single-sided planar configuration is used. In the sensor assembly 900, a three-electrode sensor is shown, with a working electrode (WE) 924, a counter electrode (CE) 926 and a reference electrode (RE) 940. In sensor assembly 900, the electrodes are co-planar. In one or more implementations, an underlay configuration of single sided co-planar analyte sensor assembly 900 or other sensor assemblies as discussed herein can enable one or more of a reference electrode (e.g., 940) or a counter electrode (e.g., 926) to be moved off of the sensor assembly 900 or other sensor assembly as discussed herein to a position outside the body (e.g., as part of a wearable device such as a smartwatch)-rather than be deployed subcutaneously. This has the advantage of freeing up space in the host (e.g., in a wound pocket where the sensor assembly 900 is inserted), where the single sided co-planar analyte sensor assembly 900 or other sensor assemblies is deployed, for an additional working electrode and/or for detecting an additional analyte.

Moreover, by configuring a wearable device as discussed herein to include the reference (940) and counter (926) electrodes, such that those electrodes are externally deployed, an amount of material inserted into the body is limited. As a result, a foreign body response of a host, e.g., the response the host's immune system, can be reduced. In one or more configurations, for instance, reference electrodes (940) can include or otherwise be formed of silver chloride (AgCl). Some hosts can have sensitivity issues with silver chloride, however. Thus, configuring the wearable device as discussed herein to include the reference electrode (940) rather than incorporating the reference electrode (940) as part of the in vivo portion of sensor assemblies discussed herein can reduce an immune response of such hosts, such as to reduce eye and/or skin irritation.

FIGS. 9A-9D depict top-down schematic views of the sensor assembly 900 being produced. FIGS. 9E-9G depict cross-sectional schematic views of the sensor assembly 900 at varying points along the length of the sensor assembly 900.

The sensor assembly 900 can extend between the first end 912 and the second end 914 and be substantially planar along its length, as measured from the first end 912 to the second end 914. The first end 912 can be, for example, a connection end, such as for allowing electrical connection of the sensor assembly 900 to a reader, computer, or other component for interpretation of signals detected with the sensor assembly 900. The first end 912 can host one or more connector pads 922.

The second end 914 can be, for example, a sensing end, for connection with or implantation in a patient, such as for detecting glucose or other analytes. The second end 914 can host the electrodes 924, 926, and 940. The second end 914 can be the implantable portion of the sensor assembly 900. The first end 912 of the sensor that has the connector pads 922 can be the proximal end of the sensor assembly 900. The second end 914 with the implantable portion of the sensor that contains the sensing electrodes can be the distal end of the sensor assembly 900.

Shown in FIG. 9A, the substrate 910 can extend between the first end 912 and the second end 914. The substrate 910 can be a relatively planar material, for example, the substrate 910 can be a thin flexible layer for hosting the other components. In some cases, the substrate 910 can be a polymeric film, such as liquid crystal polymer (LCP), polyimide (PI), polyethylene terephthalate (PET), combinations thereof, or similar polymeric films. The substrate 910 can have a thickness of about 25 to about 450 μm, such as a thickness of about 75 to 100 μm. In some examples, a substrate thickness of about 40 μm to about 80 μm can be used.

The conductive traces 921, connector pads 922, working electrode 924, and counter electrode 926 can be made from a conductive layer 920 built on the substrate. The connector pads 922 can be situated on or at the first end 912 of the assembly 900 and allow for electrical connection of the sensor assembly 900. The working electrode 924 and the counter electrode 926 can be sensing electrodes exposed at the second end 914 of the assembly 900 for implantation and sensing of an analyte in a patient environment. The conductive traces 921 can connected the electrodes 924, 926, to the connector pads 922.

Shown in FIG. 9B, the conductive layer 920 can be built up on the substrate 910 with the conductive traces 921, connector pads 922, working electrode 924, and counter electrode 926 in a single plane or layer. The conductive layer 920 can, for example, be made of a sputtered metal, such as titanium/gold/platinum or platinum/gold/platinum sputtered metal layers. In this case, relevant sensing surfaces such as at the working electrode 924 can have exposed platinum for electrical connection and sensing. The reference electrode 940 can be deposited on a base metal pad, and can be connected through additional conductive traces.

In some examples, the conductive layer 920 is formed from a single conductor, such as gold or platinum. In other examples, the conductive layer 920 or can be formed from more than one material, such as a thin palladium layer that is covered with gold and platinum. The composition, geometry, and exposed conductor surfaces can depend on the manufacturing method, desired mechanical properties, and requirements of the sensing chemistry. For example, the base conductive material can be formed by a less expensive material, such as silver, which is covered in strategic locations by platinum for the active sensing surfaces. In some cases, gold can be plated as the base conductor, which can be covered with platinum in order to provide both mechanical robustness and an active sensing surface for sensing hydrogen peroxide.

The conductive layer 920, including the working electrode 924, counter electrode 926, connector pads 922, and conductive traces 921, can be formed by a variety of techniques, such as plating, sputtering, or printing. To form the structure of patterning of the conductive layers, standard photolithographic techniques, laser ablation, or printing (e.g., inkjet or screen printing) can be used.

Although certain electrode designations are shown in the supporting document, it should be understood that the size, shape, and electrode identity can be changed depending on a specific use case, such as a particular analyte to be determined. The general size and shape of the sensor is 3 mm to 4 mm wide at the proximal end (connector end) and 300-500 μm wide in the narrow implantable distal end. The overall length of the sensor is dependent on the requirements of the wearable/inserter but are generally between 15 mm and 25 mm.

Shown in FIG. 9C, the insulator 930 can be layered on top of the conductive layer 920 as desired. Insulating materials can be referred to as “solder mask,” “dielectric,” or “insulator.” These materials can be used to protect the conductive traces from exposure to the sample matrix and environment, as well as improve the accuracy and reliability of measurements by defining the sensing electrode area. An opening 931 can be made for later deposition of the reference electrode 940.

Here, the insulator 930 can be made of an electrically insulating material deposited on top of the conductive layer to protect the conductive traces 921 and define the openings for the connector pads 922, and the electrodes 924, 926, in addition to an opening 931 for the reference electrode 940. The insulator 930 can be, for example, a thin layer of solder mask.

Shown in FIG. 9D, the reference electrode 940 material can be deposited over the designated reference electrode opening in the insulator 930. The reference electrode 940 material can be, for example, a silver/silver chloride formulation. It can be deposited on the designated sensing electrode pad. This reference electrode material can be deposited by a printing technique, such as screen printing, or by discrete dispense, such as a jet-valve dispenser.

FIGS. 9E-9G depict cross-section of the assembly 900 at varying points along the body of the assembly. FIG. 9E shows a cross-section at line E-E of FIG. 9D, in a central portion of the assembly 900. At this part of the assembly 900, the conductive traces 921 can be seen between the insulator 930 and the substrate 910. FIG. 9F shows a cross-section at line F-F of FIG. 9D, near the second end 914 of the assembly 900. At this part of the assembly 900, the reference electrode 940 can be seen on top of the conductive traces 921. FIG. 9G depicts a cross-section at line G-G of the assembly, near the second end 914. Here, the working electrode 924 can be seen. The assembly 900 is a single sided, co-planar arrangement for the electrodes 924, 926, 940.

### Methods for Determining Glucose and 1,5-AG Levels from Glucose and Pyranose Sensors

As described herein, the continuous analyte monitoring system 104 comprises one or more single analyte sensors, and/or a multi-analyte sensor in a co-axial or co-planar configuration. In certain embodiments, continuous analyte monitoring system monitors a patient's glucose levels and pyranose levels, to determine the patient's 1,5-AG levels. Because a continuous pyranose sensor device captures the level of all pyranose sugars in the body, including glucose and 1,5-AG (among a few other negligible pyranose sugars), the measured current on the pyranose-selective electrode would represent a summation of glucose and 1,5-AG levels of the patient. While glucose and 1,5-AG comprise a majority of pyranose sugars, it is understood that additional pyranose sugars are also present in minor concentrations, but minor concentrations of additional pyranose sugars are generally not expected to result in interference of the accuracy of the measurement of 1,5-AG levels.

In an embodiment with one or more single analyte sensors, to determine the real-time 1,5-AG levels of the patient, the signal at the continuous glucose sensor device is subtracted from the signal at the continuous pyranose sensor device. The result of this subtraction is the signal representative of 1,5-AG levels and, therefore, the 1,5-AG levels can be inferred.

In an embodiment with a multi-analyte sensor, to determine the real-time 1,5-AG levels of the patient, the signal at the glucose-selective electrode is subtracted from the signal at the pyranose-selective electrode. The result of this subtraction is the signal representative of 1,5-AG levels and, therefore, the 1,5-AG levels can be inferred.

In certain embodiments, the differential between the signal from the glucose-selective electrode and the pyranose-selective electrode is calculated in analog hardware or digital hardware. For example, using analog hardware, the analog signal from the glucose-selective electrode ise subtracted from the analog signal from the pyranose-selective electrode using a differential mode amplifier, difference amplifier, or subtractor (or similar analog device or circuit). The differential mode amplifier creates an output analog signal to be converted into a digital signal.

Where the differential is calculated using digital hardware, the two analog signals from the glucose-selective electrode and the pyranose-selective electrode are digitized independently and subtracted from one another by means of a digital subtractor. Further, in the digitized form, the signals from the glucose-selective electrode and the pyranose-selective electrode are subtracted from one another in firmware and/or software (e.g., by means of an embedded algorithm carrying out the mathematical function of pyranose-selective sensor signal value minus glucose-selective sensor signal value).

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases can refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. A nalyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein can refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein can refer to two or more system elements or components that are configured to be or have been electrically, mechanically, thermally, operably, chemically, or otherwise attached and detached without damaging any of the coupled elements or components. The phrase “permanently coupled” as used herein can refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), transcutaneously (i.e. penetrating, entering, or passing through intact skin), or subcutaneously (i.e., penetrating to the subcutis or adipose tissue region under the skin), which can result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface. Interfering species can feature their own electroactive nature or otherwise inhibit or activate an enzyme present in a sensor that partakes in the transduction operation. Interfering species can also contribute to changes in one or more of the sensing membranes present, thereby adjusting the flux of the desired analyte(s).

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators can be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, service as an interface to attenuate the foreign body response/fibrous encapsulation, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

A II numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

### Example Embodiments

Implementation examples are described in the following numbered clauses:

Clause 1: A monitoring system, comprising: one or more memories comprising executable instructions; and one or more processors in data communication with the one or more memories and configured to execute the executable instructions to: calculate a first reabsorption threshold based on the glucose measurements and the 1,5-AG measurements of the patient over a first period of time, wherein: an analog to digital converter is configured to receive a second sensor current and convert the second sensor current generated by the continuous analyte sensor into a second set of digital signals; and a processor is configured to convert the second set of digital signals to a second set of analyte measurements indicative of a second set of analyte levels of the patient, wherein the second set of analyte measurements include a second set of glucose measurements and a second set of 1,5-AG measurements; the one or more processors are further configured to: calculate a second reabsorption threshold based on the second set of glucose measurements and the second set of 1,5-AG measurements over a second period of time.

Clause 2: A monitoring system, comprising: one or more memories comprising executable instructions; and one or more processors in data communication with the one or more memories and configured to execute the executable instructions to: calculate a first reabsorption threshold based on glucose measurements and 1,5-AG measurements of a patient over a first period of time; calculate a second reabsorption threshold based on the glucose measurements and the 1,5-AG measurements of the patient over a second period of time; detect a change of the second reabsorption threshold relative to the first reabsorption threshold; determine whether the change of the second reabsorption threshold relative to the first reabsorption threshold is an increase or a decrease; and provide therapy management guidance to the patient based on the increase or the decrease.

Clause 3: A monitoring system, comprising: one or more memories comprising executable instructions; and one or more processors in data communication with the one or more memories and configured to execute the executable instructions to: detect a decline in 1,5-AG levels of a patient; initiate a monitoring period at time TO; determine a first level of 1,5-AG at time T0; monitor 1,5-AG levels of the patient on a periodic basis; determine a second level of 1,5-AG at time T1; calculate a value representing a total cleared 1,5-AG; calculate a total mass of 1,5-AG cleared; determine a filtration score of the patient; and provide therapy management guidance to the patient based on the filtration score of the patient.

Clause 4: The monitoring system of any one of Clauses 1-3, wherein the monitoring system further comprises: a continuous analyte sensor configured to penetrate a skin of a patient and generate sensor current indicative of analyte levels of the patient; a sensor electronics module coupled to the continuous analyte sensor, wherein the sensor electronics module comprises: an analog to digital converter configured to: receive the sensor current; and convert the sensor current generated by the continuous analyte sensor into digital signals; a processor configured to convert the digital signals to a set of analyte measurements indicative of the analyte levels of the patient; and a Bluetooth antenna configured to transmit the set of analyte measurements wirelessly to a wireless communications device using Bluetooth or BLE communications protocols.

Clause 5: The monitoring system of any one of Clauses 1-4, wherein the sensor electronic module further comprises a sensitivity profile for the monitoring system based on a calibration process performed during manufacturing, wherein the processor being configured to convert the digital signals to the set of analyte measurements comprises the processor being configured to convert the digital signals to the set of analyte measurements based on the sensitivity profile.

Clause 6: The monitoring system of any one of Clauses 1-5, wherein the continuous analyte sensor comprises: a percutaneous wire comprising: a proximal portion coupled to the sensor electronics module; and a distal portion comprising a working electrode and a reference electrode, wherein the working electrode is configured to penetrate the skin and extend into a dermis or subcutaneous tissue of the patient.

Clause 7: The monitoring system of any one of Clauses 1-6, wherein: the working electrode and the reference electrode are disposed on a substrate, and the sensor current is at least in part based on a voltage difference generated between the working electrode and the reference electrode.

Clause 8: The monitoring system of any one of Clauses 1-7, wherein: the continuous analyte sensor is a multi-analyte sensor comprising a continuous glucose sensor and a continuous pyranose sensor, and the set of analyte measurements include glucose measurements and 1,5-AG measurements, wherein the 1,5-AG measurements are determined by subtracting a signal from the continuous glucose sensor from a signal from the continuous pyranose sensor.

Clause 9: The monitoring system of any one of Clauses 1-2 or 4-8, wherein the one or more processors are further configured to detect a change of the second reabsorption threshold relative to the first reabsorption threshold by comparing the second reabsorption threshold to the first reabsorption threshold.

Clause 10: The monitoring system of any one of Clauses 1-2 or 4-9, wherein the one or more processors are further configured to determine whether the change of the second reabsorption threshold relative to the first reabsorption threshold is an increase or a decrease.

Clause 11: The monitoring system of any one of Clauses 1-2 or 4-10, wherein, based on a determination that the change of the second reabsorption threshold relative to the first reabsorption threshold is an increase, the one or more processors are further configured to determine a manner in which the increase occurred.

Clause 12: The monitoring system of any one of Clauses 1-2 or 4-11, wherein the determination of the manner in which the increase occurred comprises determining (1) whether the increase occurred following a decrease in a reabsorption threshold of the patient prior to the calculation of the first reabsorption threshold or (2) whether the increase is in response to the reabsorption threshold of the patient prior to the calculation of the first reabsorption threshold being below a defined threshold.

Clause 13: The monitoring system of any one of Clauses 1-2 or 4-12, wherein, if the increase did not follow the decrease in the reabsorption threshold and the increase is not in response to the reabsorption threshold being below the defined threshold, the one or more processors are further configured to provide therapy management guidance to the patient to seek medical intervention for a decline in kidney function.

Clause 14: The monitoring system of any one of Clauses 1-2 or 4-13, wherein, if the increase followed the decrease in the reabsorption threshold or the increase is in response to the reabsorption threshold below the defined threshold, the one or more processors are further configured to continue calculating one or more subsequent reabsorption thresholds for the patient.

Clause 15: The monitoring system of any one of Clauses 1-2 or 4-14, wherein, based on a determination that the change of the second reabsorption threshold relative to the first reabsorption threshold is a decrease, the one or more processors are further configured to determine a manner in which the decrease occurred.

Clause 16: The monitoring system of any one of Clauses 1-2 or 4-15, wherein the determination of the manner in which the decrease occurred comprises determining (1) a rate of change of the reabsorption threshold between the first reabsorption threshold and the second reabsorption threshold or (2) a decrease in one or more reabsorption thresholds of the patient over subsequent periods of time prior to the calculation of the first reabsorption threshold.

Clause 17: The monitoring system of any one of Clauses 1-2 or 4-16, wherein, if the rate of change of the reabsorption threshold between the first reabsorption threshold and the second reabsorption threshold is above a defined threshold rate of change, the one or more processors are further configured to provide therapy management guidance to the patient to seek medical intervention for acute kidney injury.

Clause 18: The monitoring system of any one of Clauses 1-2 or 4-17, wherein, if there is a decrease in the one or more reabsorption thresholds of the patient over subsequent periods of time, the one or more processors are further configured to provide therapy management guidance to the patient to seek medical intervention for late stage chronic kidney failure.

Clause 19: The monitoring system of any one of Clauses 1-2 or 4-18, wherein the determination of the manner in which the decrease occurred comprises determining (1) whether the decrease occurred following a increase in reabsorption threshold of the patient prior to the calculation of the first reabsorption threshold, or (2) whether the decrease is in response to the reabsorption threshold of the patient prior to the calculation of the first reabsorption threshold being above a defined threshold.

Clause 20: The monitoring system of any one of Clauses 1-2 or 4-19, wherein if the decrease followed the increase in the reabsorption threshold or the decrease is in response to the reabsorption threshold being above the defined threshold, the one or more processors are further configured to notify the patient of an improvement in kidney function and continue calculating one or more subsequent reabsorption thresholds for the patient,

Clause 21: The monitoring system of any one of Clauses 1-2 or 4-20, wherein the one or more processors are further configured to determine whether the patient is taking an SGLT2 inhibitor.

Clause 22: The monitoring system of any one of Clauses 1-2 or 4-21, wherein the one or more processors are further configured to, based on the glucose measurements and the 1,5-AG measurements, determine whether the glucose measurements and the 1,5-AG measurements are within a defined range and provide guidance to the patient to reach the defined range.

Clause 23: The monitoring system of any one of Clauses 3-8, wherein the one or more processors configured to detect the decline in the 1,5-AG levels of the patient comprises the one or more processors being configured to detect the decline in the 1,5-AG levels of the patient based on the patient reaching a specified reabsorption threshold, a downward trend in the 1,5-AG levels, or a negative rate of change of 1,5-AG levels.

Clause 24: The monitoring system of any one of Clauses 3-8 or 23, wherein the one or more processors are further configured to initiate a monitoring period at the time T₀.

Clause 25: The monitoring system of any one of Clauses 3-8 or 23-24, wherein the one or more processors configured to determine the second level of 1,5-AG at the time T₁ comprises the one or more processors being configured to determine the second level of 1,5-AG at the time T₁ based on a determination that a predetermined amount of 1,5-AG has cleared from the body.

Clause 26: The monitoring system of any one of Clauses 3-8 or 23-25, wherein the time T₁ is a time at which the 1,5-AG level of the patient meets a threshold level indicative of 1,5-AG being cleared from the body or a time at which a determined rate of change of decline of 1,5-AG is below a threshold rate of change indicative of 1,5-AG clearance slowing or stopping.

Clause 27: The monitoring system of any one of Clauses 3-8 or 23-26, wherein the time T₁ is a time at which the glucose levels of the patient reach a predefined level or a time at which glucose levels begin to decrease towards a reabsorption threshold of the patient.

Clause 28: The monitoring system of any one of Clauses 3-8 or 23-27, wherein the time T₁ is a predefined time after T₀.

Clause 29: The monitoring system of any one of Clauses 3-8 or 23-28, wherein the determination of the first level of 1,5-AG at the time T₀ and the second level of 1,5-AG at the time T₁ are based on urine samples of the patient.

Clause 30: The monitoring system of any one of Clauses 3-8 or 23-29, wherein the determination of the total volume of 1,5-AG cleared is based on a urine concentration of 1,5-AG at time T₁ multiplied by a volume of urine collected in the urine sample of the patient.

Clause 31: The monitoring system of any one of Clauses 3-8 or 23-30, wherein the determination of the total volume of 1,5-AG cleared is based on the first level of 1,5-AG at time T₀ minus the second level of 1,5-AG at time T₁ multiplied by the total body water volume of the patient.

Clause 32: The monitoring system of any one of Clauses 3-8 or 23-31, wherein the total body water volume is based on a body mass of the patient, an age of the patient, and a gender of the patient.

Clause 33: The monitoring system of any one of Clauses 3-8 or 23-32, wherein the one or more processors are further configured to provide therapy management guidance to the patient based on the filtration score of the patient.

Clause 34: The monitoring system of any one of Clauses 3-8 or 23-33, wherein the filtration score of the patient is provided to the patient via a display device.

Clause 35: The monitoring system of any one of Clauses 3-8 or 23-34, wherein, based on a detected increase in the filtration score of the patient over time, the one or more processors are further configured to provide therapy management guidance to the patient that the kidney function of the patient is improving.

Clause 36: The monitoring system of any one of Clauses 3-8 or 23-35, wherein, based on a detected decrease in the filtration score of the patient over time, the one or more processors are further configured to provide therapy management guidance to the patient that the kidney function of the patient has worsened.

Clause 37: The monitoring system of any one of Clauses 3-8 or 23-36, wherein the one or more processors are further configured to, based on the glucose measurements and the 1,5-AG measurements, determine whether the glucose measurements and the 1,5-AG measurements are within a defined range and provide guidance to the patient to reach the defined range.

Clause 38: A method for performing operations performed by the system in Clauses 1-37.

Clause 39: A device configured to perform operations performed by the system in Clauses 1-37.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to penetrate a skin of a patient and generate sensor current indicative of analyte levels of the patient;
a sensor electronics module coupled to the continuous analyte sensor, wherein the sensor electronics module comprises:
an analog to digital converter configured to:
receive the sensor current; and
convert the sensor current generated by the continuous analyte sensor into digital signals;
a processor configured to convert the digital signals to a set of analyte measurements indicative of the analyte levels of the patient; and
a Bluetooth antenna configured to transmit the set of analyte measurements wirelessly to a wireless communications device using Bluetooth or BLE communications protocols.

2. The monitoring system of claim 1, wherein the sensor electronic module further comprises a sensitivity profile for the monitoring system based on a calibration process performed during manufacturing, wherein the processor being configured to convert the digital signals to the set of analyte measurements comprises the processor being configured to convert the digital signals to the set of analyte measurements based on the sensitivity profile.

3. The monitoring system of claim 1, wherein the continuous analyte sensor comprises:
a percutaneous wire comprising:
a proximal portion coupled to the sensor electronics module; and
a distal portion comprising a working electrode and a reference electrode, wherein the working electrode is configured to penetrate the skin and extend into a dermis or subcutaneous tissue of the patient.

4. The monitoring system of claim 3, wherein:
the working electrode and the reference electrode are disposed on a substrate, and
the sensor current is at least in part based on a voltage difference generated between the working electrode and the reference electrode.

5. The monitoring system of claim 1, wherein:
the continuous analyte sensor is a multi-analyte sensor comprising a continuous glucose sensor and a continuous pyranose sensor, and
the set of analyte measurements include glucose measurements and 1,5-AG measurements, wherein the 1,5-AG measurements are determined by subtracting a signal from the continuous glucose sensor from a signal from the continuous pyranose sensor.

6. The monitoring system of claim 5, further comprising:
one or more memories comprising executable instructions; and
one or more processors in data communication with the one or more memories and configured to execute the executable instructions to:
calculate a first reabsorption threshold based on the glucose measurements and the 1,5-AG measurements of the patient over a first period of time, wherein:
the analog to digital converter is further configured to receive a second sensor current and convert the second sensor current generated by the continuous analyte sensor into a second set of digital signals; and
the processor is further configured to convert the second set of digital signals to a second set of analyte measurements indicative of a second set of analyte levels of the patient, wherein the second set of analyte measurements include a second set of glucose measurements and a second set of 1,5-AG measurements;
the one or more processors are further configured to:
calculate a second reabsorption threshold based on the second set of glucose measurements and the second set of 1,5-AG measurements over a second period of time.

7. The monitoring system of claim 6, wherein the one or more processors are further configured to detect a change of the second reabsorption threshold relative to the first reabsorption threshold by comparing the second reabsorption threshold to the first reabsorption threshold.

8. The monitoring system of claim 7, wherein the one or more processors are further configured to determine whether the change of the second reabsorption threshold relative to the first reabsorption threshold is an increase or a decrease.

9. The monitoring system of claim 8, wherein, based on a determination that the change of the second reabsorption threshold relative to the first reabsorption threshold is an increase, the one or more processors are further configured to determine a manner in which the increase occurred.

10. The monitoring system of claim 9, wherein the determination of the manner in which the increase occurred comprises determining (1) whether the increase occurred following a decrease in a reabsorption threshold of the patient prior to the calculation of the first reabsorption threshold or (2) whether the increase is in response to the reabsorption threshold of the patient prior to the calculation of the first reabsorption threshold being below a defined threshold.

11. The monitoring system of claim 10, wherein, if the increase did not follow the decrease in the reabsorption threshold and the increase is not in response to the reabsorption threshold being below the defined threshold, the one or more processors are further configured to provide therapy management guidance to the patient to seek medical intervention for a decline in kidney function.

12. The monitoring system of claim 10, wherein, if the increase followed the decrease in the reabsorption threshold or the increase is in response to the reabsorption threshold below the defined threshold, the one or more processors are further configured to continue calculating one or more subsequent reabsorption thresholds for the patient.

13. The monitoring system of claim 8, wherein, based on a determination that the change of the second reabsorption threshold relative to the first reabsorption threshold is a decrease, the one or more processors are further configured to determine a manner in which the decrease occurred.

14. The monitoring system of claim 13, wherein the determination of the manner in which the decrease occurred comprises determining (1) a rate of change of the reabsorption threshold between the first reabsorption threshold and the second reabsorption threshold or (2) a decrease in one or more reabsorption thresholds of the patient over subsequent periods of time prior to the calculation of the first reabsorption threshold.

15. The monitoring system of claim 14, wherein, if the rate of change of the reabsorption threshold between the first reabsorption threshold and the second reabsorption threshold is above a defined threshold rate of change, the one or more processors are further configured to provide therapy management guidance to the patient to seek medical intervention for acute kidney injury.

16. The monitoring system of claim 14, wherein, if there is a decrease in the one or more reabsorption thresholds of the patient over subsequent periods of time, the one or more processors are further configured to provide therapy management guidance to the patient to seek medical intervention for late stage chronic kidney failure.

17. The monitoring system of claim 13, wherein the determination of the manner in which the decrease occurred comprises determining (1) whether the decrease occurred following a increase in reabsorption threshold of the patient prior to the calculation of the first reabsorption threshold, or (2) whether the decrease is in response to the reabsorption threshold of the patient prior to the calculation of the first reabsorption threshold being above a defined threshold.

18. The monitoring system of claim 17, wherein if the decrease followed the increase in the reabsorption threshold or the decrease is in response to the reabsorption threshold being above the defined threshold, the one or more processors are further configured to notify the patient of an improvement in kidney function and continue calculating one or more subsequent reabsorption thresholds for the patient.

19. The monitoring system of claim 6, wherein the one or more processors are further configured to determine whether the patient is taking an SGLT2 inhibitor.

20. The monitoring system of claim 6, wherein the one or more processors are further configured to, based on the glucose measurements and the 1,5-AG measurements, determine whether the glucose measurements and the 1,5-AG measurements are within a defined range and provide guidance to the patient to reach the defined range.
